SEQUENCE LISTING (47 sequences)

SEQ ID NO: 1 - DNA, 23 bp, Artificial Sequence
  Synthetic Oligonucleotide
ttaatctctcncgtagcggagnt

SEQ ID NO: 2 - DNA, 23 bp, Artificial Sequence
  Synthetic Oligonucleotide
atnttntcgtcgccgatgaagnc

SEQ ID NO: 3 - DNA, 23 bp, Artificial Sequence
  Synthetic Oligonucleotide
gccttcatcgncgacnacaacat

SEQ ID NO: 4 - DNA, 23 bp, Artificial Sequence
  Synthetic Oligonucleotide
ctttnacagataacganaaggtc

SEQ ID NO: 5 - DNA, 23 bp, Artificial Sequence
  Synthetic Oligonucleotide
ctttcacanataacgacaangtc

SEQ ID NO: 6 - DNA, 23 bp, Artificial Sequence
  Synthetic Oligonucleotide
ctanccagagagctcccagnctc

SEQ ID NO: 7 - DNA, 23 bp, Artificial Sequence
  Synthetic Oligonucleotide
tttcttntgaaagaaacttgnca

SEQ ID NO: 8 - DNA, 23 bp, Artificial Sequence
  Synthetic Oligonucleotide
ctagccagagagctcccaggctc

SEQ ID NO: 9 - DNA, 23 bp, Artificial Sequence
  Synthetic Oligonucleotide
tttcttgtgaaagaaacttggca

SEQ ID NO: 10 - DNA, 23 bp, Artificial Sequence
  Synthetic Oligonucleotide
tggagacagcatgcgaacgttta

SEQ ID NO: 11 - DNA, 23 bp, Artificial Sequence
  Synthetic Oligonucleotide
taaacgttcgcatgctgtctcca

SEQ ID NO: 12 - DNA, 23 bp, Artificial Sequence
  Synthetic Oligonucleotide
taaangttcgcatgctgtctcca

SEQ ID NO: 13 - DNA, 23 bp, Artificial Sequence
  Synthetic Oligonucleotide
taaacnttcgcatgctgtctcca

SEQ ID NO: 14 - DNA, 23 bp, Artificial Sequence
  Synthetic Oligonucleotide
ctttnacagataacganaaggtc

SEQ ID NO: 15 - DNA, 23 bp, Artificial Sequence
  Synthetic Oligonucleotide
ctttcacanataacgacaangtc

SEQ ID NO: 16 - DNA, 23 bp, Artificial Sequence
  Synthetic Oligonucleotide
gggcgggtccccagggggggcag

SEQ ID NO: 17 - DNA, 23 bp, Artificial Sequence
  Synthetic Oligonucleotide
gggcgngtccccagggggggcag

SEQ ID NO: 18 - DNA, 23 bp, Artificial Sequence
  Synthetic Oligonucleotide
ggncnngtccccagggnnggcag

SEQ ID NO: 19 - DNA, 23 bp, Artificial Sequence
  Synthetic Oligonucleotide
gnncnnntccccannnnggncag

SEQ ID NO: 20 - DNA, 23 bp, Artificial Sequence
  Synthetic Oligonucleotide
nnncnnntccccannnnnnncag

SEQ ID NO: 21 - DNA, 23 bp, Artificial Sequence
  Synthetic Oligonucleotide
ctgcacagccccctcccctgggc

SEQ ID NO: 22 - DNA, 23 bp, Artificial Sequence
  Synthetic Oligonucleotide
ctgnacagccccctcccctgggc

SEQ ID NO: 23 - DNA, 23 bp, Artificial Sequence
  Synthetic Oligonucleotide
ctgnacagnnccctcnnctgggc

SEQ ID NO: 24 - DNA, 23 bp, Artificial Sequence
  Synthetic Oligonucleotide
ctgnacagnnnnntnnnntgggc

SEQ ID NO: 25 - DNA, 23 bp, Artificial Sequence
  Synthetic Oligonucleotide
ntgnanagnnnnntnnnntgggc

SEQ ID NO: 26 - DNA, 23 bp, Artificial Sequence
  Synthetic Oligonucleotide
agttgtctcctgcctgcttagtc

SEQ ID NO: 27 - DNA, 23 bp, Artificial Sequence
  Synthetic Oligonucleotide
agnngncnccngccngcnnagnc

SEQ ID NO: 28 - DNA, 23 bp, Artificial Sequence
  Synthetic Oligonucleotide
agttgtntnntgnntgnttagtn

SEQ ID NO: 29 - DNA, 23 bp, Artificial Sequence
  Synthetic Oligonucleotide
agttgtntnntgnntgnttagtn

SEQ ID NO: 30 - DNA, 23 bp, Artificial Sequence
  Sythetic Oligonucleotide
anttntctcctncctncttantc

SEQ ID NO: 31 - DNA, 23 bp, Artificial Sequence
  Synthetic Oligonucleotide
anttntctcctncctncttantc

SEQ ID NO: 32 - DNA, 23 bp, Artificial Sequence
  Sythetic Oligonucleotide
agttgtntnntgnntgnttagtn

SEQ ID NO: 33 - DNA, 23 bp, Artificial Sequence
  Synthetic Oligonucleotide
agttgtntnntgnntgnttagtn

SEQ ID NO: 34 - DNA, 23 bp, Artificial Sequence
  Synthetic Oligonucleotide
agttgtntnntgnntgnttagtn

SEQ ID NO: 35 - DNA, 23 bp, Artificial Sequence
  Synthetic Oligonucleotide
anttntntnntnnntnnttantn

SEQ ID NO: 36 - DNA, 23 bp, Artificial Sequence
  Synthetic Oligonucleotide
anttntntnntnnntnnttantn

SEQ ID NO: 37 - DNA, 23 bp, Artificial Sequence
  Synethetic Oligonucleotide
agnngnnnnnngnnngnnnagnn

SEQ ID NO: 38 - DNA, 23 bp, Artificial Sequence
  Synthetic Oligonucleotide
agnngncnccngccngcnnagnc

SEQ ID NO: 39 - DNA, 23 bp, Artificial Sequence
  Synthetic Oligonucleotide
agttgtntnntgnntgnttagtn

SEQ ID NO: 40 - DNA, 23 bp, Artificial Sequence
  Synthetic Oligonucleotide
agttgtntnntgnntgnttagtn

SEQ ID NO: 41 - DNA, 23 bp, Artificial Sequence
  Synthetic Oligonucleotide
anttntctcctncctncttantc

SEQ ID NO: 42 - DNA, 23 bp, Artificial Sequence
  Synthetic Oligonucleotide
anttntctcctncctncttantc

SEQ ID NO: 43 - DNA, 23 bp, Artificial Sequence
  Synthetic Oligonucleotide
agttgtntnntgnntgnttagtn

SEQ ID NO: 44 - DNA, 23 bp, Artificial Sequence
  Synthetic Oligonucleotide
agttgtntnntgnntnnttagtn

SEQ ID NO: 45 - DNA, 23 bp, Artificial Sequence
  Synthetic Oligonucleotide
anttntntnntnnntnnttnntn

SEQ ID NO: 46 - DNA, 23 bp, Artificial Sequence
  Synthetic Oligonucleotide
anttntntnntnnntnnttnntn

SEQ ID NO: 47 - DNA, 23 bp, Artificial Sequence
  Synthetic Oligonucleotides
agnngnnnnnngnnngnnnagnn

(19) United States
(12) Patent Application Publication
(10) Pub. No.: US 2011/0171287 A1
(43) Pub. Date: Jul. 14, 2011

(54) Use of Oligonucleotides with Modified Bases as Antiviral Agents

(75) Inventors: Mart Saarma, Helsinki (FI); Andres Merits, Tartu (EE); Mati Karelson, Tartu (EE)

(73) Assignee: BALTIC TECHNOLOGY DEVELPMENT, LTD., Tallinn (EE)

(21) Appl. No.: 12/741,531

(22) PCT Filed: Nov. 5, 2008
(86) PCT No.: PCT/FI2008/050633

§ 371 Date: Feb. 4, 2011

Related U.S. Application Data
(60) Provisional application No. 60/985,548, filed on Nov. 5, 2007.
(60) Provisional application No. 61/057,685, filed on May 30, 2008.

(51) Int. Cl. A61K 9/127; A61K 31/7115; C07H 21/00; C12N 9/96; C12Q 1/70; C12N 5/07; A61P 31/12; A61P 31/14; A61P 31/20

(52) U.S. Cl. 424/450; 514/44 R; 536/23.1; 435/188; 435/5; 435/375

(57) ABSTRACT

The present invention relates to the use of oligonucleotides having modified nucleobases to inhibit gene expression and/or replication of viruses in a subject. The modified nucleobases may be mercapto-modified bases or hydroxy-modified nucleobases. It is contemplated that the oligonucleotides further comprise a nuclease complex which enhances anti-viral activity of the oligonucleotides.

### CROSS-REFERENCE TO RELATED APPLICATIONS

This application claims the benefit of prior U.S. provisional application Nos. 61/057,685 filed on May 30, 2008 and 60/985,548 filed on Nov. 5, 2007, each of which is hereby incorporated by reference in its entirety.

### FIELD OF THE INVENTION

The present invention relates to use of oligonucleotide analogs that contain specifically modified nucleotide bases to inhibit gene expression and/or replication of viruses. The oligonucleotides are optionally bound to organic complexes of lanthanides with highly selective artificial nuclease activity.

### BACKGROUND OF THE INVENTION

The use of oligonucleotides and modified oligonucleotides is of great significance in modern therapy and has been well documented (Uhlmann, et al. Antisense oligonucleotides: A new therapeutic principle. Chemical Reviews 1990, 90: 543-584; Crooke, et al. “Antisense Research and Applications”, CRC Press (1993); Mesmaekar, et al. “Antisense oligonucleotides,” Acc. Chem. Res. 1995, 28: 366-374; Stein. “The experimental use of antisense oligonucleotides: a guide for the perplexed.” J. Clin. Invest. 2001, 108, 641-644). The specific binding of antisense polynucleotides to the DNA or RNA targets can inactivate the replication, transcription, or translation of nucleic acids, thereby providing a mechanism for controlling diseases such as cancer and viral infection. The binding of an antisense oligonucleotide to a target can thus be used to alter gene expression, in a variety of circumstances, e.g., to interfere with viral life cycles, or the growth of cancerous cells.

Many important infectious diseases afflicting mankind are caused by viruses. Many of these diseases, including hepatitis, immune deficiencies and various encephalitic diseases, are frequently fatal. Others are significant in that they are highly contagious and create acute discomfort such as influenza, measles, mumps and chickenpox, as well as respiratory or gastrointestinal disorders. Others such as rubella and cytomegalovirus can cause congenital abnormalities. Finally there are viruses, known as oncoviruses (including human papillomaviruses), which can cause cancer in humans and animals.

Viral genomes can consist of either DNA or RNA. In addition, two groups of viruses, which use both DNA and RNA phases during their replication cycle, are known. Viral genomic DNA may be double-stranded or single-stranded, circular or linear. The size of the DNA can be as small as 4.5 kb or as big as 1.2 Mbp; the number of genes varies from 3 to up to 911. DNA genomes do not serve as targets for suggested antivirals. Instead the virus encoded mRNAs, micro-RNAs as well as mRNAs encoding host-factors, crucial for virus infection, may be used as antisense targets. Accordingly, this approach will not lead to direct destruction of the viral genome, but can suppress virus replication and/or gene expression and eventually lead to the clearance of the virus genome from the cells. Additionally, targeting of viral mRNAs encoding proteins which are essential for suppression of host immune responses may enhance virus elimination by host immune system. Targeting of the viral gene products essential for inhibition of host cell apoptosis may lead to premature death of infected cells and thus eliminate the virus infection, while targeting of virus gene products essential for maintenance of latent viruses (mRNAs or in some cases micro-RNAs) may lead to elimination of latently infected cells and removal of latent virus from the infected organism.

Retroviruses do not have a DNA genome, but at the first stage of their infection they synthesize the cDNA copy of their genomic RNA, which is then integrated into the cellular DNA and thus becomes a part of the host genome (so called provirus DNA). This DNA is often transcriptionally silent and cells carrying these proviruses cannot be targeted by conventional anti-viral agents or systems (this is one of the biggest problems in anti-HIV therapy). Although the provirus cannot be targeted or eliminated by antisense technology, certain steps of the infection process can. These steps include: targeting of the viral genome in newly infected cells before the reverse transcription reaction takes place—the genomic RNA at this stage is enclosed with viral proteins in a “core” particle; targeting of viral receptors or co-receptors (in case of HIV chemokine receptors) to prevent virus entry; targeting of cellular cofactors essential for virus cDNA nuclear transport and integration in host genome (a number of such factors are known for HIV-1); targeting of the mRNAs expressed from provirus and thus suppressing the retrovirus replication cycle; targeting of the viral genomic RNAs before their encapsidation and preventing formation of new virions. These strategies may be efficient for inhibiting the HIV genome, which encodes multiple regulatory proteins for which expression can be blocked.

Genomes of riboviruses (viruses with RNA genomes) can be directly targeted with sequence-specific anti-RNA agents. Therefore the infection caused by riboviruses can be totally eliminated by use of these kinds of drugs alone. In practice, however, the targeting of the genomes may be complicated since the viral genomes are often protected; but even then the viral mRNAs and mRNAs of cellular cofactors essential for the virus may be targeted.

The genomic RNA of viruses with double-stranded RNA genomes is always packed in protein particles (viral core) and is never released to the cytoplasm of an infected cell. Thus, most likely only mRNAs and host factors can be targeted with anti-RNA agents. The genome of viruses with single-stranded negative RNA genomes is always protected by viral nucleoprotein, but it does not form a specific core-particle. Thus, it would be possible to target both the virus genomes as well as their essential mRNAs.

Viruses with single-stranded positive RNA genomes are the only viruses in which the genomic RNA is directly used as mRNA and is, at least at some stage of infection, in naked (unprotected) form. However, during the replication, the genome and its complementary strand are usually packed in membranous structures. Therefore it is possible that the initial stages of infection (before formation of the replicase complex) may be more sensitive to the RNA degrading agents. In addition to the nature of the viral target (genome, mRNA or both) additional factors may have very important roles as well.

The abundance of target RNA is especially important if the antiviral agent is limited to a single action such as blocking one RNA molecule (antisense oligonucleotide) or cleaving just one RNA molecule (this is often the case with anti-viral ribozymes). The abundance of viral RNA varies greatly depending on the type of virus and the stage of infection. It is hypothesized that antisense agents are more efficient at the stages of the infection where the copy-number of viral RNA (genomes or mRNAs) is low (typically this corresponds to the early stages of infection).

Polarity of the target is important for inhibition of positive-sense riboviruses, which have highly asymmetric RNA synthesis, e.g., the number of positive (genomic) strands of these viruses is often 100 or more times higher than that of negative strands. This would make the negative strand a preferable target, however it is known that the negative strands of these viruses are often (possibly always) presented in form of a double-stranded RNA intermediate and hidden in membranous replication complexes. It would, therefore, be important to determine if the proposed antiviral agent also binds to dsRNA complexes (specific degradation of negative strands has been reported in experiments using RNAi).

The site of virus replication inside of the infected cell depends on the type of the virus and can be carried out in the nucleus (retroviruses, most DNA viruses and influenza virus) or cytoplasm (all riboviruses, except influenza viruses, bornaviruses and nucleorhabdoviruses and DNA-genomic poxviruses). For viruses replicating inside the cytoplasm, different virus-specific structures (often named “virus factories” or “viroplasms”) are observed. In theory the use of specific sites (e.g., nucleus, membrane complexes, and “virus factories”) can protect viral genomes from therapeutic agents; nevertheless it has been reported that at least RNAi is active against nuclear RNA molecules. Thus, the importance of the intracellular localization of viral replication complexes may have a relatively minor effect, especially if the following considerations are taken into account: all viruses use free mRNAs to express their genes and thus have molecules which can be targeted by the RNA degrading agent(s), and all positive strand RNA viruses (the only viruses whose genomes can be easily targeted) replicate in the cytoplasm.

The site of virus replication inside an infected organism likely has much greater impact. Again, the site of replication depends from the type of the virus. Most of the viruses have certain tissue-specificity—some infect epithelial cells, some viruses infect hepatocytes, etc. Many viruses infect different cell types. The infection may start from peripheral tissues (portal of entry), but the disease is caused due to replication in other cell type(s) (target cells). There are a number of viruses where the two cells are rather different (measles virus etc), but there are also many examples where these cells are identical (influenza virus).

The consequences of the different replication sites inside the organism vary. If the virus replicates in epithelial tissues (gut epithelial cells, respiratory tract) the methods of drug delivery can be relatively simple. This means that effective but highly unstable compounds like siRNAs can be used to treat these infections in vivo. If the virus replicates in tissues other than epithelia, a specific drug-delivery system is needed at least for siRNA technology. In the case of HCV infection the therapeutic agent can theoretically be liver-specific nanoparticles or liver-specific viral particles expressing siRNA precursors (shRNA). In transient models the injection of plasmid DNA encoding shRNA directly into the bloodstream has also produced good results, but therapeutic use of such technology would most likely be limited due to the lack of specificity of the delivery. Compounds that are stable enough to be delivered by blood and safe and non-toxic (e.g., short oligonucleotides cannot integrate into host DNA and generally do not cause side effects) could have advantages over siRNA technology in these viruses.

If the virus replicates in specific tissues, like neuronal tissue, another type of specific delivery system may be needed. If the target tissue of the virus is CNS, then a method to cross the blood-brain barrier, such as intracerebral delivery of the antiviral agent, is needed as well.

Viruses infecting humans and animals generally use one of several infection strategies. In the “hit and run” strategy (acute infections), the virus enters the host, replicates quickly and actively and is ultimately eliminated by the host immune response; the only alternative is the death of the infected host. Infection time is typically short (up to a few weeks) and viral amounts during acute infection are very high. This strategy is used by many “classical” viruses (as example—influenza virus).

In the “hit and hide” strategy (latent infections), the infection starts and proceeds as in the above scenario, but instead of complete elimination, virus establishes life-long latent infection. During latency viral gene expression is minimized and no infectious virus is produced. However, under certain conditions virus re-activates and causes new disease (either similar or different from the initial one). These types of viruses are very common and include, for example herpes simplex virus (HSV). In many cases these viruses present serious problems due to the tendency to cause severe or fatal diseases in immuno compromised patients (such as AIDS patients).

A “chronic” or “persistent” infection generally starts with a short acute phase. This phase can be asymptomatic and can end with complete elimination of virus. The outcome, however, depends on both virus and host: up to 95% of hepatitis B virus (HBV) infections in adults end with elimination of virus. Alternatively, virus may establish chronic infection (for example, up to 70% of HCV infections and possibly 100% of HIV infections). During chronic infection the viral replication remains active (in contrast to that in latent infection) and a large amount of virions may be produced (HCV typically forms up to 10¹² particles per day). Nevertheless the immune system is unable to eliminate the infection. Chronic viruses represent a major healthcare problem since up to 500 million people are infected with HBV or HCV and around 40 million people are infected with HIV-1.

The requirements for elimination of different types of infection may be drastically different. With acute diseases the treatment must be done as early as possible and most likely should be rather short. In this case the treatment has a clear aim, slow down the virus infection and give the immune system time to eliminate the virus (complete virus inhibition by treatment may not be possible and in many cases may not be needed). It is not clear what should be the minimal efficiency of the used agent—in some cases a minor inhibition of replication may provide considerable protection, in other cases almost complete inhibition may be required.

With latent viruses the major problem is the elimination of the latently infected cells—a reservoir, from where new infections may start. Currently the mechanisms of viral latency are poorly understood and that is one of the reasons why current treatments cannot eliminate the latent virus. In theory, these viruses and latent cells can be targeted with sequence-specific RNA degrading agents against viral cofactors (if they are known) or against essential viral gene products. For example, in a study of HSV it was recently demonstrated that the virus encoded micro-RNA may be the main factor responsible for latent infection, which may provide a desirable target for inhibition of viral replication.

With chronic viruses the treatment may aim toward either prevention of the spread of infection (for example in case of liver transplantation to HCV patients), to eliminate the infection in all or in many cells or, at least, to keep virus replication under some kind of control. Generally, the treatment of chronic infection is a long process—currently life-long for HIV patients and for many months for HBV and HCV patients. Due to this fact the side effects of the treatment are very important as is seen in long term effects with anti-HIV, -HCV and -HBV treatment.

One of the main problems with all types of antiviral agents is the appearance of viral mutants resistant to the drug. The general molecular mechanisms behind this phenomenon include deletion of the target sequences and modification (point mutations, small deletions) of the target sequence. In many cases the complete gene or its function may be lost (anti-HSV therapy with acyclovir often leads to the loss of viral TK gene). However, this mechanism of resistance is possible only if the target gene encodes a function that is non-essential for viral survival. Modifications of the sequences outside of the actual target so that they compensate for the lost function, facilitate the change of RNA conformation etc, are problematic for consistent anti-viral treatment. Another issue is the fact that many important pathogens are genetically extremely variable. The existing variation of the genomic sequences of HIV-1 is comparable with that in whole Picornaviridae family; the homology between HCV genotypes can be as low as 60%. In essence, this indicates that neither HIV-1 nor HCV represent a single pathogen; instead they are names used (largely for historical reasons) for groups of related pathogens. This huge diversity has seriously hampered the construction of efficient vaccines against either of these viruses. Furthermore, the HCV, HIV as well as other riboviruses do not have a fixed genome sequence as such, instead they have a sort of consensus sequence (individual genomes generally differ for that) and therefore exist as quasi-species. It has been shown that even inside of one single patient the individual sequences of HCV RNAs differ from each other in 1-3% of nucleotide positions. Thus, considerable sequence variability already exists in any chronically infected patient.

These sequence variations often result from mutations taking place during viral replications and recombination events. Mutations during viral replication primarily happen due to the lack of proofreading function—neither reverse transcriptase (HIV-1) nor RNA dependent RNA polymerase (HCV) is capable of correcting the errors that occur during viral replication (the error rate is estimated 10⁻⁴ changes per nucleotide per cycle, in the case of HIV and HCV it means one change per genome). Taken together with the large amount of genomes synthesized by these viruses, both HIV and HCV are capable of mutating extremely fast (HCV is reported to have a mutation rate about 100 higher than HIV). Essentially this means that these viruses can overcome the effects of any sequence-specific therapeutic agent in a short period of time. This phenomenon has been most intensively studied for HIV, since anti-HIV therapy has been used over nearly 20 years. The results indicate that in order to obtain any long term effect the inhibitors with different target specificity should be used in combinations (this is the principle of current Highly Active Anti Retrovirus Therapy: HAART). However even this strategy does not prevent the appearance of new drug-resistant HIV variants.

The phenomenon of resistance of viruses to nucleic acid based therapies (antisense oligonucleotides, ribozymes, siRNA and shRNA) is most studied for the RNAi system. It has been demonstrated that both retro-(HIV) and riboviruses (HCV, poliovirus) are sensitive to RNAi, but they rapidly develop resistance against any specific siRNA by introduction of mutations in the siRNA target site and/or surrounding sequences. It has also been shown that the resistance is always connected with specific target sequence and not with insensitivity to the RNAi as such. Development of the resistance against siRNAs is apparently rather easy, since siRNA should have 100% match with the target (micro-RNA tolerates several changes, but is a far less efficient silencer). Further, due to the redundancy of the genetic code the nucleic acid sequence can change without affecting the sequence of encoded protein. Viral proteins do also have considerable plasticity—many amino-acid changes are tolerated (this is evident from the data about the anti-HIV drug resistance as well). Changes can affect not only RNA sequence but also its secondary structure and thus inhibit its recognition by siRNA.

There are two general approaches how to avoid (or minimize) the appearance of resistant viral genomes. Similar to HAART therapy, several siRNAs (or a combination of siRNAs with other inhibitors such as ribozymes) can be used to treat viral infection. It has been shown that such treatment is more effective than treatment with a single siRNA and significantly reduces the appearance of resistant mutants. Therefore a similar strategy should be strongly recommended for other nucleic acid based therapeutic agents as well.

siRNAs can be targeted to highly conserved sequences or those having multiple overlapping functions. Such sequences cannot be easily changed without affecting crucial functions. Examples of such sequences are primer binding regions in the HIV-1 genome or 5′ UTR region with IRES element in the HCV genome.

### SUMMARY OF THE INVENTION

The present invention relates to compositions comprising oligonucleotides wherein the oligonucleotides comprise modified nucleobases, and optionally chelating moieties, which increase their binding ability to complementary nucleic acids and exhibit antiviral activity.

In one aspect, the invention provides a method of inhibiting replication of a virus in a subject comprising administering to the subject an oligonucleotide having from 5 to 150 nucleobases, wherein at least one nucleobase is a mercapto-modified tautomeric or ionic base (mercaptonucleobase) or a hydroxyl-modified tautomeric or ionic base (hydroxynucelobase).

In another aspect, the invention contemplates a method of inhibiting translation of a target nucleic acid comprising contacting the target nucleic acid with a composition comprising an oligonucleotide having from 5 to 150 nucleobases under conditions that permit hybridizing of the oligonucleotide to the target nucleic acid, wherein the hybridized oligonucleotide inhibits translation of the target nucleic acid, wherein the target nucleic acid is associated with viral replication, and wherein the oligonucleotide comprises at least one modified nucleobase selected from the group consisting of a mercapto-modified tautomeric or ionic base (mercaptonucleobase) and a hydroxy-modified tautomeric or ionic base (hydroxynucleobase).

In a further aspect, the invention provides a method of inhibiting replication of a viral genome in a subject or viral pathogen, comprising predicting or determining a nucleotide sequence of a target nucleic acid in a subject, wherein the target nucleic acid is associated with viral replication, and administering to the subject a composition comprising an oligonucleotide having from 5 to 150 nucleobases, wherein at least one nucleobase is a mercapto-modified tautomeric or ionic base (mercaptonucleobase) or a hydroxy-modified tautomeric or ionic base (hydroxynucleobase), and wherein under physiological conditions of the subject, said compound is sufficiently complementary to the nucleotide sequence of the target sequence to hybridize thereto in the subject and inhibit viral replication.

The invention further contemplates use of an oligonucleotide to inhibt viral replication in a subject, wherein the oligonucletide is formulated for administration to the subject and comprises from 5 to 150 nucleobases, wherein at least one nucleobase is a mercapto-modified tautomeric or ionic base (mercaptonucleobase) or a hydroxy-modified tautomeric or ionic base (hydroxynucleobase).

In a related aspect, the invention provides for use of an oligonucleotide in the manufacture of a medicament to inhibit translation of a target nucleic acid, wherein the target nucleic acid is contacted with a composition comprising an oligonucleotide under conditions that permit hybridizing of the oligonucleotide to the target nucleic acid, wherein the hybridized oligonucleotide inhibits translation of the target nucleic acid, wherein the target nucleic acid is associated with viral replication, and wherein the oligonucleotide comprises at least one modified nucleobase selected from the group consisting of a mercapto-modified tautomeric or ionic base (mercaptonucleobase) and a hydroxy-modified tautomeric or ionic base (hydroxynucleobase).

In a further aspect, the invention provides for use of an oligonucleotide in the manufacture of a medicament to inhibit replication of a viral genome in a subject, the use comprising predicting or determining a nucleotide sequence of a target nucleic acid in a subject or a viral pathogen, wherein the target nucleic acid is associated with viral replication, and administering to the subject a medicament comprising an oligonucleotide having from 5 to 150 nucleobases, wherein at least one nucleobase is a mercapto-modified tautomeric or ionic base (mercaptonucleobase) or a hydroxy-modified tautomeric or ionic base (hydroxynucleobase), and wherein under physiological conditions of the subject, said compound is sufficiently complementary to the nucleotide sequence of the target sequence to hybridize thereto in the subject and inhibit viral replication.

In one embodiment, the oligonucleotide useful in The method or use or compound of the invention comprises at least one mercaptonucleobase. In a related embodiment, the mercaptonucleobases is selected from the group consisting of 5-mercaptocytosine, 5-mercaptouracil, 8-mercaptoguanine and 8-mercaptoadenine. In a related embodiment, the invention comprises at least one hydroxynucleobase. In a still further embodiment, the hydroxynucleobase is selected from the group consisting of 5-hydroxycytosine, 5-hydroxyuracil, 8-hydroxyadenine and 8-hydroxyguanine.

In one aspect, the oligonucleotide further comprises an organic nuclease attached thereto. In one embodiment, the organic nuclease comprises a chelating organic moiety complexed with a lanthanide metal. In a preferred embodiment, the lanthanide metal is selected from the group consisting of lanthanum, cerium, praseodymium, neodymium, promethium, samarium, europium, gadolinium, terbium, dysprosium, holmium, erbium, thulium, ytterbium, and lutetium.

In a further aspect, the oligonucleotides described herein are useful to inhibit replication of various virus strains. In one embodiment, the virus is a positive-strand RNA virus. In a related embodiment, the virus is a DNA virus.

In a still further embodiment, the virus is a fast acute virus. In a related embodiment, the fast acute virus is an alphavirus.

In another embodiment, the virus is a chronic virus. In a related embodiment, the chronic virus is hepatitis C virus.

In yet another embodiment, the virus is a papillomavirus. In a related embodiment, the papilloma virus is selected from the group consisting of bovine papilloma virus 1 and human papilloma virus.

In one embodiment of the method or use contemplated above, the oligonucleotide hybridizes to a viral gene encoding a transcription or regulatory factor, and inhibits replication of a virus with a DNA genome. In a related embodiment, the oligonucleotide hybridizes to a viral replication factor and inhibits replication of a virus with a DNA genome.

In a further embodiment, the oligonucleotide hybridizes to a virus using reverse transcription in its replication cycle. In yet another embodiment, the oligonucleotide hybridizes to a viral gene encoding a transcription or a regulatory factor to inhibit replication of a virus replicating by use of reverse transcription. In a preferred embodiment, the virus using reverse transcription is a retrovirus. In a more preferred embodiment, the retrovirus is human immunodeficiency virus 1.

In a related embodiment, the oligonucleotide hybridizes to a host factor associated with viral replication, and inhibits viral replication. In a still further embodiment, the oligonucleotides are useful to treat uninfected cells to decrease the viral gene expression and replication in these cells.

It is contemplated that the oligonucleotide useful in the methods of the invention are from 5 to 150 nucleobases in length, from 10 to 100 nucleobases in length, 10 to 50 nucleobases in length, from 10 to 30 nucleobases in length, from 20 to 30 nucleobases in length, or from 21 to 23 nucleobases in length. All integer lengths from 5 to 150 nucleobases, and also subranges within 5 to 150 nucleobases, are specifically contemplated for practice of the invention.

It is further contemplated that 1% to 100% of nucleobases in the modified oligonucleotide are modified nucleobases. In one embodiment, 10% to 90% of the nucleobases are modified nucleobases. In a related embodiment, 20% to 80% of the nucleobases are modified nucleobases. In a further embodiment, 30% to 70%, 40% to 60% or 50% of the nucleobases are modified nucleobases. In a still further embodiment, 10, 20 30, 40, 50, 60, 70, 80 or 90% of the nucleobases are modified nucleobases.

In a related embodiment, the oligonucleotide comprises 1, 2, 3, 4, 5, 6, 7, 8, 9, 10, 11, 12, 13, 14, 15, 16, 17, 18, 19, 20, up to 30, up to 40, up to 50, up to 100 or more modified nucleobases, wherein the modified nucleobase is a hydroxynucleobase or a mercaptonucleobase. For example, it is contemplated that the entirety of a 150-base oligonucleotide may comprise all modified oligonucleotides.

In an embodiment of the invention, the oligonucleotide hybridizes to a non-coding region of a viral genome or hybridizes to a coding region of a viral genome. In a related embodiment, the oligonucleotide hybridizes to the coding region of an RNA virus.

The method or use or compound of the invention also provides that, in one aspect, at least two oligonucleotides having different sequences are administered to the subject, wherein the oligonucleotides hybridize to different target sequences. The invention further provides for use of at least two oligonucleotides having different sequences in the manufacture of a medicament for administration to a subject, wherein the oligonucleotides hybridize to different target sequences. In one embodiment, two different oligonucleotides are administered to the subject. In a further embodiment, the oligonucleotides are specific for different target sequences in the same viral genome. In another embodiment, the oligonucleotides are specific for target sequences in the same functional unit. In yet another embodiment, the oligonucleotides are specific for target sequences in different functional units.

It is further contemplated that the composition further comprises a pharmaceutical carrier or excipient as described herein.

In one embodiment, the subject is a mammal. In a related embodiment, the subject is human.

In one aspect, the composition for use in The method or use or compound of the invention is administered in a liposome. In a related embodiment, the composition may be administered in a nanoparticulate pharmaceutical such as a micelle or a nanoparticle.

It is contemplated that the hybridizing of the oligonucleotide to a target sequence induces cleavage of the target nucleic acid.

In one embodiment, the oligonucleotide exhibits at least 70% homology to the target nucleic acid. In a related embodiment, the oligonucleotide is 75%, 80%, 85%, 90%, 91%, 92%, 93%, 94%, 95%, 96%, 97%, 98% or 99% homologous to the target nucleic acid.

In addition to the foregoing, the invention includes, as an additional aspect, all embodiments of the invention narrower in scope in any way than the variations specifically mentioned above. For example, although aspects of the invention may have been described by reference to a genus or a range of values for brevity, it should be understood that each member of the genus and each value or sub-range within the range is intended as an aspect of the invention. Likewise, various aspects and features of the invention can be combined, creating additional aspects which are intended to be within the scope of the invention.

Aspects of the invention described in the singular (including the use of articles “a” or “an” should be understood to include embodiments involving one or more than one, unless context clearly requires a narrower interpretation. The term “comprising” is intended to be permissive of additional elements or features.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows the suppression of E2 dependent replication of BPV origin containing plasmids by antisense modified oligonucleotides targeted against E2 mRNA.

FIG. 2 illustrates the effect of antisense oligonucleotides with modified 5-OH-dC bases on the replication of recombinant virus Inhibition of replication of recombinant virus is monitored by measuring Rluc activity expressed from genomic RNA of the virus LF—lipofectamine treated control cells. Results from one of the reproducible experiments are shown in the figure.

FIG. 3 shows the additive effect of the nuclease complex on the antiviral effect of oligonucleotides with modified 8-oxo-dG bases Inhibition of replication of recombinant virus is monitored by measuring Rluc activity expressed from genomic RNA of the virus.

FIG. 4 demonstrates the reduction of effective dose 50 (EC50) for HMO_{—}9 by the presence of the nuclease (compound designated HMO 9E).

### DETAILED DESCRIPTION OF THE INVENTION

This present invention relates to the use of oligonucleotides containing one or more modified bases, which optionally may be linked to an organic nuclease complex, for suppression of the replication and/or gene expression of viruses pathogenic to humans and animals.

The current invention provides compounds that comprise a chelating moiety and an oligonucleotide having properties for use in antisense inhibition of viral gene expression and/or replication. The compounds of the invention include antisense oligonucleotides having (a) one or more modified nucleobases having high binding efficiency to natural nucleobases and, optionally, (b) one chelating moiety. These compounds can hydrolyze phosphodiester bonds of oligonucleotides, RNA, and/or DNA, and are useful in antisense therapies.

### Viral Targets

Examples of important viral pathogens belonging to different systemic groups, exploiting different replication strategies and different target tissues and problems with anti-viral therapy against these viruses are described below.

AIDS (acquired immunodeficiency syndrome) is caused by the human immunodeficiency virus (HIV). By killing or damaging cells of the body's immune system, HIV progressively destroys the body's ability to fight infections and certain cancers. There are currently approximately 42 million people living with HIV/AIDS worldwide. A total of 3.1 million people died of HIV/AIDS related causes in 2002. The ultimate goal of anti-HIV drug therapy is to prevent the virus from reproducing and damaging the immune system. Although substantial progress has been made over the past fifteen years in the fight against HIV, a cure still eludes medical science. Today, physicians have more than a dozen antiretroviral agents in three different drug classes to manage the disease. Typically, drugs from two or three classes are prescribed in a variety of combinations known as HAART (Highly Active AntiRetroviral Treatment). HAART therapies typically comprise two nucleoside reverse transcriptase inhibitors drugs with a third drug, either a protease inhibitor or a non-nucleoside reverse transcriptase inhibitor. Clinical studies have shown that HAART is the most effective means of reducing viral loads and minimizing the likelihood of drug resistance.

There is a great need for the development of other antiviral agents effective against HIV that work through other mechanisms of action against which the virus has not developed resistance. This is becoming especially important in view of recent data showing that 1 out of 10 patients newly diagnosed with HIV in Europe is infected with a strain of HIV already resistant to at least one of the approved drugs on the market.

Hepatitis C virus (HCV) infection is the most common chronic bloodborne infection in the United States where the number of infected patients likely exceeds 4 million and 150 million worldwide. This common viral infection is a leading cause of cirrhosis and liver cancer, and is now the leading reason for liver transplantation in the United States. Recovery from infection is uncommon, and about 85 percent of infected patients become chronic carriers of the virus and 10 to 20 percent develop cirrhosis. It is estimated that there are currently 170 million people worldwide who are chronic carriers. According to the Centers for Disease Control and Prevention, chronic hepatitis C causes between 8,000 and 10,000 deaths and leads to about 1,000 liver transplants in the United States alone each year. There is no vaccine available for hepatitis C. Prolonged therapy with interferon alpha, or the combination of interferon with Ribavirin, is effective in only about 40 percent of patients and causes significant side effects.

There are over one hundred species of human infecting viruses (human papillomaviruses, HPVs) in this family. HPV causes benign skin warts, or papillomas. Many HPVs are transmitted through sexual contact. Genital HPV infection is very common, with estimates suggesting that up to 75% of women will become infected with one or more of the sexually transmitted HPV types at some point during adulthood. Persistent infection with “high-risk” sexually transmitted HPVs (HPV-16, -18 and some others) can lead to the development of cancer of the cervix. Thus HPVs are medically important (up to 500,000 cancer cases/year). In 2006/2007, effective vaccines became available against the four most prevalent HPV species, however a number of HPV strains and species are not covered by these vaccines.

Alphaviruses are representatives of the viruses which are causing mostly acute infections. More than 10 members of this genus are known human pathogens. They are often widespread but their medical importance was considered to be moderate, and as such, there is no effective vaccine for humans against alphaviruses. The dramatic outbreak of Chikungunya virus on Reunion Island and in India radically changed this view. The overall economic damage caused by Chikungunya virus on Reunion Island alone is estimated to be over 100 million euros (235,000 infections, ca 200 death cases), the damage caused by this virus in India is taking the scale of a national medical disaster (estimates are as high as 6 million infections). Chikungunya virus is still rapidly spreading and in late 2007 a mosquito-born outbreak was first detected in Europe (Italy). Taking into account the unpredictable nature of outbreaks caused by alphaviruses, large scale vaccination would not appear to be practical as there is no way to predict which virus will emerge and where the next outbreak will occur. Instead an anti-viral therapy, ideally effective against many alphaviruses, would be more useful.

The viruses listed above represent only a fraction of the known viral pathogens for humans and domestic animals, however they essentially cover all variety of the different viral genome types (DNA, RNA, retroviral); target tissue (epithelia, immune system, liver specificity and viruses with a broad range of target cells) and replication sites (cytoplasm or nucleus), specificity and the type of the infection (acute, chronic or latent). These viruses also represent typical viruses causing cell death (alphaviruses, HIV), no apparent cell damage (HCV) or cell transformation (papillomaviruses). Thus, the data obtained by studies of these representative viruses can be extended to all known types of viral pathogens for humans and animals.

It is clear that for any new emerging antiviral drug being developed, it would be highly desirable to incorporate the three following features: (1) improved efficacy; (2) reduced risks of side effects, and (3) a mechanism of action which is difficult for the virus to overcome by mutation.

Attempts to inhibit particular viruses by various antisense approaches have been made.

Zamecnik et al. have used oligonucleotides (ONs) specifically targeted to the reverse transcriptase primer site and to splice donor/acceptor sites (Zamecnik, et al (1986) Proc. Natl. Acad. Sci. USA 83:4143) (Goodchild & Zamecnik (1989) U.S. Pat. No. 4,806,463).

Oligonucleotides or oligonucleotide analogs targeting CMV (cytomegalovirus) mRNAs coding for IE1, IE2 or DNA polymerase were reported by Anderson et al (1997) (U.S. Pat. No. 5,591,720).

Hanecak et al (1999) (U.S. Pat. No. 5,952,490) have described modified oligonucleotides having a conserved G quartet sequence and a sufficient number of flanking nucleotides to significantly inhibit the activity of a virus such as HSV-1 (herpes simplec virus).

Qi et al. (Zhonghua Shi Yan He Lin Chuang Bing Du Xue Za Zhi (2000) 14:253-256) have reported testing antisense phosphorothioate-oligonucleotides (PS-ONs) in Coxsackie virus B3.

International publication WO 9203051 (Roizman and Maxwell) describes methylphosphonate antisense oligomers which are complementary to vital regions of HSV viral genome or mRNA transcripts thereof which exhibit antiviral activity.

Guanosine/thymidine or guanosine-rich phosphorothioate oligodeoxynucleotides (GT-PS—ONs) have been reported to have antiviral activity. The study reported that “several different PS-containing GT-rich ONs (B106-140, 1100-12, and G106-57) all 26 or 27 nt in length, were just as effective at reducing HIV-2 titers as GT-rich ONs consisting of 36 (B106-96, B106-97) or 45 nt (Table 4).” (Fennewald et al., Antiviral Res. (1995) 26:37-54).

Cohen et al. (U.S. Pat. Nos. 5,264,423 and 5,276,019) described the inhibition of replication of HIV, and more particularly to PS-ODN (oligodeoxynucleotide) analogs that can be used to prevent replication of foreign nucleic acids in the presence of normal living cells. Cohen et al. describe antiviral activity of antisense PS-ODNs specific to a viral sequence. They also describe testing polyA, polyT and polyC PS-ODN sequences of 14, 18, 21 and 28-mers and indicate an antiviral effect of those PS-ODNs.

Gao et al ((1989) J Biol Chem 264:11521-11526), describe the inhibition of replication of HSV-2, by PS-ODNs by testing of polyA, polyT and polyC PS-ODN sequences in sizes of 7, 15, 21 and 28 nucleotides.

Stein & Cheng (Stein et al. (1993) Science 261:1004-1012), discuss the antiviral activity of non specific ODNs of 28 nucleotides, stating that “the anti-HIV properties of PS oligos are significantly influenced by non-sequence-specific effects, that is, the inhibitory effect is independent of the base sequence.”

In a review article, Lebedeva & Stein (Lebedeva et al (2001) Annul Rev Pharmacol 41:403-419) report a variety of non-specific protein binding activity of PS-ODNs, including viral proteins. They state that “these molecules are highly biologically active, and it is often relatively easy to mistake artifact for antisense.”

Rein et al. (U.S. Pat. No. 6,316,190) reported a GT rich ON decoy linked to a fusion partner and binding to the HIV nucleocapsid, which can be used as an antiviral compound. Similarly, Campbell et al. (Campbell et al (1999) J. Virol. 73 :2270-2279) reported PO-ODN with a TGTGT motif binding specifically to the nucleocapsid of HIV but with no references to an antiviral activity.

Antisense ODNs developed as anticancer agents, antiviral agents, or to treat others diseases are typically approximately 20 nucleotides in length. In a review article (Stein, C A, (2001) J. Clin. Invest. 108:641-644), it is affirmed that “the length of an antisense oligonucleotide must be optimized: If the antisense oligonucleotide is either too long or too short, an element of specificity is lost. At the present time, the optimal length for an antisense oligonucleotide seems to be roughly 16-20 nucleotides”. Similarly, in another review article (Crooke, S T (2000) Methods Enzymol. 313:3-45) it is stated that “Compared to RNA and RNA duplex formation, a phosphorothioate oligodeoxynucleotide has a Tₘ approximately −2.2° C. lower per unit. This means that to be effective in vitro, phosphorothioate oligodeoxynucleotides must typically be 17- to −20-mer in length . . . ”

Carruthers and co-workers (Marshall et al. (1992) Proc. Natl. Acad. Sci. USA 89:6265-6269) reported anti-HIV activity of phosphorodithioate ODNs (PS2-ODNs) for a 12-mer polycytidine-PS2-ODN and for a 14-mer PS2-ODN. No other sizes were tested for anti-HIV activity. They also reported the inhibition of HIV reverse transcriptase (RT) for 12-, 14-, 20- and 28-mer polycytidine-PS2-ODNs. Later, this group (Marshal et al (1993) Science 259:1564-1570) reported results showing sequence specific inhibition of the HIV RT. The same group published data for PS2-ODNs in several patents. In U.S. Pat. Nos. 5,218,103 and 5,684.148, P52-ODN structure and synthesis is described. In U.S. Pat. Nos. 5,452,496, 5,278,302, and 5,695,979 inhibition of HIV RT is described for P52-ODNs not longer than 15 bases. In U.S. Pat. Nos. 5,750,666 and 5,602,244, antisense activity of PS2-ODNs is described. All publications are incorporated herein by reference in their entirety.

Oligonucleotides modified at the 2′ position of the ribose and their uses in antisense strategies have been evaluated, e.g., as described in the references cited below.

Inoue and coworkers (Inoue et al. (1985) Nucleic Acids Res. 16:165168) describe the synthesis and properties of oligos (2′-O-methylribonucleotides). The same group (Inoue et al. (1987) FEBS Letter 215:327-330) reported that no RNAse H mediated mRNA cleavage occurs when the oligonucleotide contains all 2′-O-methylribonucleotides. With mixed oligonucleotides, i.e., oligonucleotides having unmodified and 2′-O-methylribonucleotides, Inoue report sequence specific RNAse H hydrolysis of the nucleic acid complex formed by RNA and 2′-O-methylribonucleotides.

Fully 2′-O-methylated and phosphorothioated oligonucleotides which do not support RNase H-mediated cleavage of target mRNA were used to determine if active antisense oligonucleotides inhibited ICAM-1 expression by an RNase H-dependent mechanism (Chiang et al., (1991) J. Biol. Chem. 266:18162-18171). They stated that these antisense oligonucleotides may be useful as therapeutic agents.

Oligonucleotides with 2′-sugar modifications including 2′-O-methyl, 2′-O-propyl, 2′-O-pentyl, and 2′-fluoro were analyzed for antisense activity. Evaluation of antisense activities of uniformly 2′-modified oligonucleotides revealed that these compounds were completely ineffective in inhibiting gene expression. Activity was restored if the compound contained a stretch of at least five 2′-deoxyribonucleotide residues. This minimum deoxyribonucleotide length correlated perfectly with the minimum length required for efficient RNase H activation in vitro. (Monia et al., 1993, J. Biol. Chem. 268:14514.)

Yu et al. ((1996) Bioorganic. Med. Chem. 4:1685-1692) reported that hybrid antisense oligonucleotides having phosphorothioate, phosphodiester, or mixed backbones with a portion of 2′-O-methyl modified sugars have a specific anti-HIV activity measured by p24 ELISA quantification.

A review article, Agrawal ((1999) Biochem. Biophys. Acta 1489:53-68) suggests that for optimum activity, antisense oligonucleotides should have a combination of various properties, instead of only increased stability toward nucleases or high affinity to target RNA. Such properties include RNAse H activation. In a later review, Agrawal and Kandimalla ((2000) Mol. Med. Today 6:72-81) say that mixed backbone oligonucleotides, including 2′-O-methyl modifications, have become the choice for second-generation antisense oligonucleotides for their improved characteristics including RNAse H activation. An antisense oligo should possess certain important characteristics such as the ability to activate RNAse H upon binding to the target RNA. (Agrawal and Kandimalla, 2001, Current Cancer Drug Target 1:197-209). For most antisense approaches, targeting RNA cleavage by RNAse H is desired in order to increase antisense potency. (Kurreck, 2003, Eur. J. Biochem. 270:1628-1644).

Many studies describe the use of the 2′-O-methoxyethyl modification in antisense oligonucleotides. An example is a study using a gapped 2′ modified oligonucleotide antisense described in Zellweger et al. ((2001) J. Pharmacol. Experimental Therapeutics 298:934-940). Another example shows inhibition of the formation of the translation initiation complex using RNase H independent 2′-O-methoxyethyl antisense. (Baker et al. (1997) J. Biol. Chem. 272 :1994-2000).

Kawasaki et al. (2003) J. Antimicrob. Chemother. 51:813-819, describes the design of a highly nuclease-resistant, dimeric hairpin guanosine-quadruplex containing 2′-O-methyl groups on the nucleosides and sulphur groups on the internucleotidic bonds, and its anti-HIV-1 activity in cultured cells.

Mou and Gray (2002) (Nucleic Acids Res. 30:749-758), indicates that, compared with typical phosphorothioate-DNA oligomers, the addition of the 2′-O-methyl modification lowers the non-specific protein binding property. The protein binding affinities of g5p for a 36-mer oligonucleotide increased in the order of dA₃₆<rA₃₆<2′-O-MeA₃₆<S-rA₃₆<<S-2′-O-Me-A₃₆<S-dA₃₆ (where d=deoxy, r=ribo, 2′-O-Me-2′-O-methyl, S=phosphorothioate). This order was in agreement with the order of S-RNA<<S-2′-O-MeRNA<S-DNA reported in Kandimalla et al. ((1998) Bioorganic Med Chem. Lett. 8:2103-2108) for the non-specific binding of plasma proteins, such as human serum albumin, gammaglobulin and fibrinogen for these oligomer modifications.

Additional knowledge exists in the art concerning the use of agents, similar to the oligonucleotides but having a different mechanism of action including small interfering RNAs (siRNAs), their precursors and analogs and ribozymes, designed to specifically bind and cleave the target nucleic acids.

Target viruses contemplated for use in the invention include, but are not limited to, positive strand RNA viruses causing acute infection (such as Semliki Forest virus, SFV, genus Alphavirus, family Togaviridae), positive strand RNA viruses causing chronic infection (such as Hepatitis C virus, HCV, genus Hepacivirus, family Flaviviridae), retroviruses causing acute infection as well as long-lasting chronic disease (such as Human Innunodeficiency virus type 1, HIV-1, genus Lentivirus, family Retroviridae) and viruses with DNA genomes (such as Bovine Papillomavirus Type 1, BPV-1, family Papillomaviridae).

The present disclosure demonstrates that a single administration of the modified oligonucleotides to cell cultures infected with these viruses, transfected with their genomes or containing their gene expression units results in specific suppression of their gene expression and (in case of RNA viruses and papillomaviruses) replication. These effects are proportional to the amount of the oligonucleotide inhibitor and increased by the presence of modified bases in the compound and further increased by the presence of the organic nuclease complex.

The present disclosure also demonstrates that the antiviral effect is increased and the effective concentration of the inhibitor is reduced if the modified bases are changed from hydroxynucleobases to mercaptonucleobases.

### Oligonucleotides

In the context of this invention, the term “oligonucleotide” refers to an oligomer or polymer of deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function in a similar manner as naturally occurring oligonucleotides when, e.g., hybridizing to target nucleic acids or interacting with complementary oligonucleotides. Such modified or substituted oligonucleotides are often preferred over native forms because of desirable properties such as, for example, enhanced cellular uptake, enhanced affinity for a target nucleic acid and increased stability in the presence of nucleases.

The efficiency of binding of compounds of the present invention to biological counterparts (e.g., RNA and/or DNA) is attained via incorporation of modified nucleobases or other analogs having zwitterionic or ionic tautomers. Compounds of the present invention have at least one nucleobase having modified nucleobases or other analogs having zwitterionic or ionic tautomers. In preferred embodiments, the modified nucleobase is a hydroxynucleobase selected from 5-hydroxycytosine and 8-hydroxyguanine or a mercaptonucleobase selected from 5-mercaptocytosine, 5-mercaptouracil, 8-mercaptoguanine, and 8-mercaptoadenine. As demonstrated in U.S. patent publication 20070259830 and WO 2007/125173, a more stable hydrogen bonding of a hydroxynucleobase or mercaptonucleobase with a nucleobase of a target nucleic acid can occur, and, therefore, those can be considered as more effectively binding to a complementary nucleic acid strand.

The acidic tautomeric group in the modified nucleobases can be any other acidic group such as the —SH, —COOH, —SO3H, etc.

In one embodiment, an oligonucleotide comprises one or more tautomeric forms of the 5-hydroxyuracil anion. In another embodiment, the compounds of the present invention include the hydroxybase 5-hydroxycytosine. In another embodiment, the hydroxybase is a tautomeric form of the 8-hydroxyadenine and its anion. Another embodiment of the invention provides compounds of the present invention modified by tautomeric forms of the 8-hydroxyguanine and its anion. Tautomeric forms of these nucleobases are described in further detail in WO 2007/125173.

Additional modified nucleobases contemplated herein include mercapto-modified nucleobases. Synthesis of mercapto-modified pyrimidines and purines is known in the art (See for example, “Chemistry of Heterocyclic Compounds: The Pyrimidines,” Supplement 1, Volume 16, Editor D. J. Brown, John Wiley & Sons, Inc., 1970, pp. 202-229; and Khalyullin et al., “Condensed purines”, Pharmaceutical Chemistry Journal, 1992, 26: 270-284). Mercaptonucleobases contemplated include 5-mercaptocytosine, 5-mercaptouracil, 8-mercaptoguanine and 8-mercaptoadenine.

Modified nucleobases have also been contemplated for use in polymerase chain reaction (PCR), hybridization of nucleic acids and siRNA-mediated gene silencing in co-owned and co-pending application Ser. No. ______ (Attorney Docket Number 28113/43435A) and U.S. provisional application 60/985,552, which are hereby incorporated by reference in its entirety.

As used herein, each of the hydroxynucleobases is considered complementary to a nucleobase when it stably hydrogen bonds to the opposite nucelobase. Therefore, in some cases, 5-hydroxyuracil is complementary to adenine, 5-hydroxycytosine is complementary to guanine, 8-hydroxyadenine is complementary to uracil and/or thymine, and 8-hydroxyguanine is complementary to cytosine. Other stable hydrogen bonding of a hydroxynucleobase with a nucleobase of a target nucleic acid can occur, and, therefore, a hydroxynucleobase is considered complementary to the nucleobases of the target nucleic acid to which stable hydrogen bonding occurs.

The number of hydroxynucleobases and/or mercaptonucleobases in a given compound of the present invention is at least 1% up to 100% of the total number of nucleobases of the oligonucleotide portion of the compound. In cases where more than one hydroxynucleobase or mercaptonucleobase is present in the compounds of the present invention, the hydroxynucleobases of mercaptonucleobases may be the same or different (in any combination of different bases and/or types of modifications). It is contemplated that 10% to 90%, 20% to 80%, 30% to 70%, 40% to 60% or 50% of the nucleobases in an oligonucleotide described herein are modified nucleobases. It is further contemplated that 10, 20, 30, 40, 50, 60, 70, 80, 90, 91, 92, 93, 94, 95, 96, 97, 98, or 99% of the nucleobases are modified nucleobases.

The compounds in accordance with this invention preferably comprise from about 5 to about 150 nucleobases (i.e. from about 5 to about 150 linked nucleosides). One of ordinary skill in the art will appreciate that the invention embodies compounds of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleobases in length.

In one preferred embodiment, the compounds of the invention are 10 to 100 nucleobases in length. One having ordinary skill in the art will appreciate that this embodies compounds of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleobases in length.

In another preferred embodiment, the compounds of the invention are 10 to 50 nucleobases in length. One having ordinary skill in the art will appreciate that this embodies compounds of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the compounds of the invention are 20 to 30 nucleobases in length. One having ordinary skill in the art will appreciate that this embodies compounds of 20, 21, 22, 23, 24, 25, 26, 27, 28, 29, or 30 nucleobases in length.

Particularly preferred compounds are oligonucleotides from about 10 to about 50 nucleobases, even more preferably those comprising from about 20 to about 30 nucleobases, the compounds used in sample tests as antiviral agents were comprised from 21 or from 23 nucleobases.

As stated above, the oligonucleotide may contain 100% modified nucleobases. As such, depending on the length of the oligonucleotide, the oligonucleotide may contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 modified nucleobases, wherein the modified base is either a mercaptonucleobase or an hydroxynucleobase.

The compound of the present invention optionally further comprises a chelating moiety. Chelating moieties function as metal ligands. They can stably chelate a metal ion. Certain metal-ligand complexes have been shown to be effective in cleaving phosphodiester bonds. In incorporating a chelating moiety into an oligonucleotide capable of antisense activity, the efficacy of the oligonucleotide in inhibiting a target nucleic acid increases due to its ability to degrade or cleave one or more phosphodiester bonds of the target nucleic acid. Therefore, the compounds of the present invention further comprise chelating moieties capable of chelating a metal ion. The metal ion is selected from the group consisting of lanthanum, cerium, praseodymium, neodymium, promethium, samarium, europium, gadolinium, terbium, dysprosium, holmium, erbium, thulium, ytterbium, and lutetium. In one aspect, preferred ions are ions of europium or lanthanum. Ions of the metals can be any stable ion, such as +1, +2, +3, +4, or +5. Preferred ions are La(III), Eu(III), Ho(III), and Ce(IV).

Contemplated chelating moieties include those represented by formulas as described below.

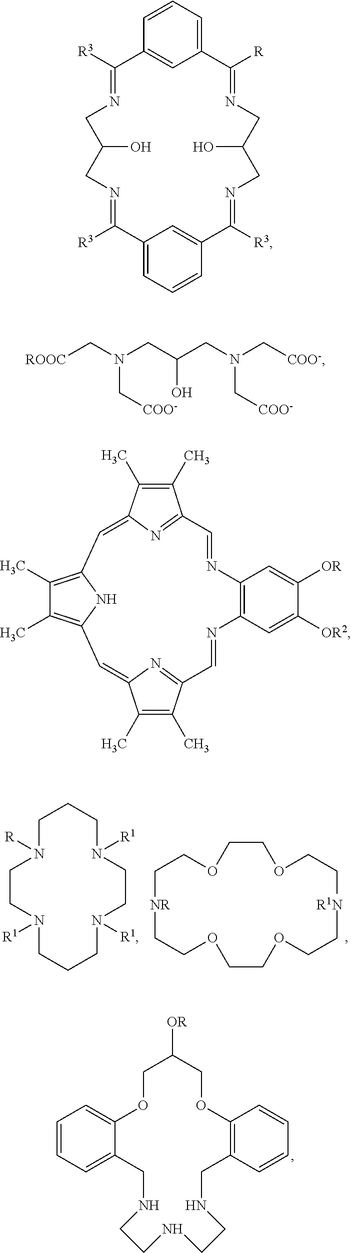

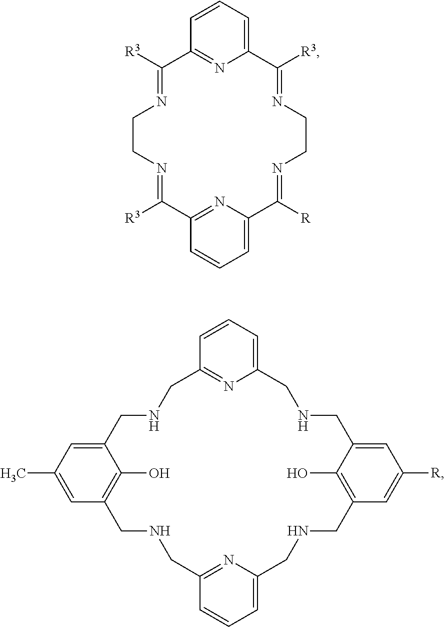

where R is the rest of the oligonucleotide;

R1 is selected from hydrogen, C1-8 alkane, C2-8 alkene, C2-8 alkyne, acylC1-8alkane, cycloalkyl, heterocycloalkyl, aryl, heteroaryl, C1-8alkylaryl, and C1-8alkylheteroaryl

R2 is independently selected from C1-8 alkyl, C2-8 alkene, C2-8 alkyne, aryl, heteroaryl, C1-8alkylaryl, C1-8alkylheteroaryl, and acylC1-8alkane, and

R3 is independently selected from the group consisting of hydrogen, C1-8 alkane, C2-8 alkene, C2-8 alkyne, acylC1-8alkane, cycloalkyl, heterocycloalkyl, aryl, heteroaryl, C1-8alkylaryl, and C1-8alkylheteroaryl.

The term “alkyl” includes straight chained and branched hydrocarbon groups containing the indicated number of carbon atoms, typically methyl, ethyl, and straight chain and branched propyl and butyl groups. The hydrocarbon group can contain up to 16 carbon atoms. The term “alkyl” includes “bridged alkyl,” e.g., a C6-C16 bicyclic or polycyclic hydrocarbon group, for example, norbornyl, adamantyl, bicyclo[2.2.2]octyl, bicyclo[2.2.1]heptyl, bicyclo[3.2.1]octyl, and decahydronaphthyl.” The term “alkyl” also encompasses alkyl groups which are optionally substituted with, e.g., one or more halogen atoms, one or more hydroxyl groups, or one or more thiol groups. The term “cycloalkyl” is defined as a cyclic C3-C8 hydrocarbon group, e.g., cyclopropyl, cyclobutyl, cyclohexyl, and cyclopentyl. “Heterocycloalkyl” is defined similar to cycloalkyl, except at least one heteroatom is present in the cyclic structure. Suitable heteroatoms include N, S, and O.

The terms “alkenyl” and “alkynyl” are defined identically as “alkyl,” except for containing a carbon-carbon double bond or carbon-carbon triple bond, respectively. “Cycloalkenyl” is defined similarly to cycloalkyl, except a carbon-carbon double bond is present in the ring.

The term “alkylene” refers to an alkyl group having a substituent. For example, the term “C1-3alkylenearyl” refers to an alkyl group containing one to three carbon atoms, and substituted with an aryl group.

The term “halo” or “halogen” is defined herein to include fluorine, bromine, chlorine, and iodine.

The term “aryl,” alone or in combination, is defined herein as a monocyclic or polycyclic aromatic group, preferably a monocyclic or bicyclic aromatic group, e.g., phenyl or naphthyl. Unless otherwise indicated, an “aryl” group can be unsubstituted or substituted, for example, with one or more, and in particular one to three, halo, alkyl, hydroxy, C(═O)OR, hydroxyalkyl, alkoxy, alkoxyalkyl, haloalkyl, haloalkoxy, cyano, nitro, amino, alkylamino, acylamino, alkylthio, alkylsulfinyl, and alkylsulfonyl. Exemplary aryl groups include phenyl, naphthyl, tetrahydronaphthyl, 2-chlorophenyl, 3-chlorophenyl, 4-chlorophenyl, 2-methylphenyl, 4-methoxyphenyl, 3-trifluoromethylphenyl, 4-nitrophenyl, and the like. The terms “arylC1-3alkyl” and “heteroarylC1-3alkyl” are defined as an aryl or heteroaryl group having a C1-3alkyl substituent.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include thienyl, furyl, pyridyl, oxazolyl, quinolyl, isoquinolyl, indolyl, triazolyl, isothiazolyl, isoxazolyl, imidazolyl, benzothiazolyl, pyrazinyl, pyrimidinyl, thiazolyl, and thiadiazolyl.

The term “Het” is defined as monocyclic, bicyclic, and tricyclic groups containing one or more heteroatoms selected from the group consisting of oxygen, nitrogen, and sulfur. A “Het” group also can contain an oxo group (═O) attached to the ring. Nonlimiting examples of Het groups include 1,3-dioxolanyl, 2-pyrazolinyl, pyrazolidinyl, pyrrolidinyl, piperazinyl, a pyrrolinyl, 2H-pyranyl, 4H-pyranyl, morpholinyl, thiopholinyl, piperidinyl, 1,4-dithianyl, and 1,4-dioxane.

The term “hydroxy” is defined as —OH.

The term “alkoxy” is defined as —OR, wherein R is alkyl.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “hydroxyalkyl” is defined as a hydroxy group appended to an alkyl group.

The term “amino” is defined as —NH₂, and the term “alkylamino” is defined as —NR2, wherein at least one R is alkyl and the second R is alkyl or hydrogen.

The term “acylamino” is defined as RC(═O)N—, wherein R is alkyl or aryl.

The term “alkylthio” is defined as —SR, wherein R is alkyl.

The term “alkylsulfinyl” is defined as RSO2-, wherein R is alkyl.

The term “alkylsulfonyl” is defined as RSO3-, wherein R is alkyl.

The term “nitro” is defined as —NO2.

The term “trifluoromethyl” is defined as —CF3.

The term “trifluoromethoxy” is defined as —OCF3.

The term “cyano” is defined as —CN.

The calculated nuclease efficiency of a compound of the present invention comprising a chelating moiety complexed to a metal ion increases, depending on the nature of the number of modified nucleobases, up to 10³-10⁹ times in comparison to naturally-occurring nucleases, allowing a corresponding lowering of the effective concentration, and keeping at the same time high specificity of the compound.

Other modifications of compounds of this invention are also contemplated. While oligonucleotides are a preferred form of the compound of the invention, the present invention comprehends other families of compounds, including, but not limited to oligonucleotide analogs and mimetics.

Additional antisense compounds contemplated for use in the compositions and methods of the invention, include but are not limited to, oligonucleotides containing modified backbones (e.g., with or without a phosphorous atom) or non-natural internucleoside linkages, oligonucleosides, modified oligonucleotide backbones that do not include a phosphorus atom which have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages (e.g., morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts), oligonucleotides having inverted polarity, oligonucleotide mimetics, optionally in which both the sugar and the internucleoside linkage (i.e. the backbone) of the nucleotide units are replaced with novel groups, peptide nucleic acid (PNA), oligonucleotides having one or more substituted sugar moieties, including but not limited to, one of the following at the 2′ position: OH; F; O—, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C10 alkyl or C2 to C10 alkenyl and alkynyl, Locked Nucleic Acids (LNAs) in which the 2′-hydroxyl group is linked to the 3′ or 4′ carbon atom of the sugar ring, thereby forming a bicyclic sugar moiety, oligonucleotides with synthetic and natural nucleobases, including but not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone, oligonucleotides chemically linked to primary or secondary hydroxyl groups, including but not limited to, chelating moieties, intercalators, reporter molecules, polyamines, polyamides, polyethylene glycols, polyethers, groups that enhance the pharmacodynamic properties of oligomers, and groups that enhance the pharmacokinetic properties of oligomers. The modifications set out above are further described in WO 2007/125173.

Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid.

### Modified Internucleoside Linkages (Backbones)

Specific examples of contemplated antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Contemplated modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Contemplated oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Contemplated modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

### Modified Sugar and Internucleoside Linkages-Mimetics

In other contemplated oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254:1497-1500.

Certain embodiments of the invention are oligonucleotides with phosphorothioate backbones and oligonucleosides with heteroatom backbones, and in particular —CH2-NH—O—CH2-, —CH2-N(CH3)-O—CH2- [known as a methylene (methylimino) or MMI backbone], —CH2-O—N(CH3)-CH2-, —CH2-N(CH3)-N(CH3)-CH2- and —O—N(CH3)-CH2-CH2- (wherein the native phosphodiester backbone is represented as —O—P—O—CH2-) of the above referenced U.S. Pat. No. 5,489,677, and the amide backbones of the above referenced U.S. Pat. No. 5,602,240. Also contemplated are oligonucleotides having morpholino backbone structures of the above-referenced U.S. Pat. No. 5,034,506.

### Modified Sugars

Modified oligonucleotides may also contain one or more substituted sugar moieties. Contemplated oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly preferred are O[(CH2)nO]mCH3, O(CH2)nOCH3, O(CH2)nNH2, O(CH2)nCH3, O(CH2)nONH2, and O(CH2)nON[(CH2)nCH3]2, where n and m are from 1 to about 10. Other preferred oligonucleotides comprise one of the following at the 2′ position: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A contemplated modification includes 2′-methoxyethoxy (2′-O-CH2CH2OCH3, also known as 2′-O-(2-methoxyethyl) or 2′-MOE) (Martin et al., Helv. Chim. Acta, 1995, 78, 486-504) i.e., an alkoxyalkoxy group. A further contemplated modification includes 2′-dimethylaminooxyethoxy, i.e., a O(CH2)2ON(CH3)2 group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH2-O—CH2-N(CH3)2, also described in examples hereinbelow.

Other contemplated modifications include 2′-methoxy (2′-O—CH3), 2′-aminopropoxy (2′-OCH2CH2CH2NH2), 2′-allyl (2′-CH2-CH═CH2), 2′-O-allyl (2′-O—CH2-CH═CH2) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methylene (—CH2-)n group bridging the 2′ oxygen atom and the 4′ carbon atom wherein n is 1 or 2. LNAs and preparation thereof are described in WO 98/39352 and WO 99/14226.

### Natural and Modified Nucleobases

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, ed. John Wiley & Sons, 1990, those disclosed by Englisch et al., Angewandte Chemie, International Edition, 1991, 30:613, and those disclosed by Sanghvi, Chapter 15, Antisense Research and Applications, pages 289-302, Crooke and Lebleu, ed., CRC Press, 1993.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, each of which is herein incorporated by reference, and U.S. Pat. No. 5,750,692, also herein incorporated by reference.

### Conjugates

Another modification of the oligonucleotides of the invention involves chemically linking to the oligonucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the invention include chelating moieties, intercalators, reporter molecules, polyamines, polyamides, polyethylene glycols, polyethers, groups that enhance the pharmacodynamic properties of oligomers, and groups that enhance the pharmacokinetic properties of oligomers.

Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application PCT/US92/09196 and U.S. Pat. No. 6,287,860, the entire disclosure of which are incorporated herein by reference.

Conjugate moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941 each of which is herein incorporated by reference.

### Antisense Inhibition

The hybridization of a compound of this invention with a target nucleic acid is generally referred to as “antisense.” Such hybridization can lead to inhibition of translation of the target nucleic acid and is termed “antisense inhibition” herein. Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be inhibited include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In the context of the present invention, “modulation” and “modulation of expression” mean either an increase (stimulation) or a decrease (inhibition) in the amount or levels of a nucleic acid molecule encoding the gene, e.g., DNA or RNA Inhibition is often the preferred form of modulation of expression and mRNA is often a preferred target nucleic acid.

In the context of this invention, “hybridization” means the pairing of complementary strands of oligomeric compounds. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In the present invention, the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated. One exemplary set of conditions is as follows: Hybridization at 42° C. in 50% formamide, 5×SSC, 20 mM Na.PO₄, pH 6.8; and washing in 1×SSC at 55° C. for 30 minutes. Formulas for calculating equivalent hybridization conditions and/or selecting other conditions to achieve a desired level of stringency are well known. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the oligonucleotide portion of the compounds of the present invention comprise at least 70% sequence complementarity to a target region within the target nucleic acid, more preferably that they comprise at least 85% or 90% sequence complementarity, and may comprise at least 95%, 96%, 97%, 98% or 99% sequence complementarity to the target region within the target nucleic acid sequence to which they are targeted. For example, a compound of the present invention in which 18 of 20 nucleobases of the compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, a compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of a compound with a region of a target nucleic acid can be determined routinely using BLAST programs (basic local alignment search tools) and PowerBLAST programs known in the art (Altschul et al., J. Mol. Biol., 1990, 215:403-410; Zhang et al., Genome Res., 1997, 7:649-656). For compounds of the present invention having hydroxynucleobases and/or synthetic analogs (such as other synthetic nucleobases), complementarity can be assessed by the synthetic analogs specificity for a particular nucleobase of the target nucleic acid.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo et al., Cell, 1995, 81:611-620). Montgomery et al. have shown that the primary interference effects of dsRNA are posttranscriptional (Montgomery et al., Proc. Natl. Acad. Sci. USA, 1998, 95:15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391:806-811). Recently, it has been shown that it is, in fact, the single-stranded RNA oligomers of antisense polarity of the dsRNAs which are the potent inducers of RNAi (Tijsterman et al., Science, 2002, 295:694-697).

### Use of Compounds of the Present Invention

The compounds described herein are used in vitro or in vivo for limiting the gene expression and proliferation of pathogens such as viruses, including viruses with DNA genomes, RNA genomes and viruses using reverse transcription. Thus, the compounds may be administered to an organism which is subject to or in a diseased state. When administered to an organism, the compounds may be used to treat infection by a variety of pathogens. As used herein “treat” refers to administration of the oligonucleotides of the invention to a subject in need in a dosage/amount sufficient to produce a desired result on a health condition, pathology, and disease of a subject or for a diagnostic purpose. The desired result may comprise a subjective or objective improvement in the recipient of the dosage. “Treatment” refers to prophylactic treatment or therapeutic treatment or diagnostic treatment. A “subject” of diagnosis or treatment is a human or non-human animal, including a mammal or a primate. A “Therapeutically effective amount” refers to that amount of a composition effective to produce the intended beneficial effect on health.

The compounds may be used to modulate the function of immune system cells such as specific B-cells; specific T-cells, such as helper cells, suppressor cells, cytotoxic T-lymphocytes (C), and natural killer (NK) cells. Modulation of immune function using the compounds of the present invention can be useful in treatment of a variety of diseases such as chronic diseases caused by viral pathogens.

The compounds may be selected which are capable of interfering with transcription and/or expression of proteins by any of the mechanisms involved with the binding of the oligonucleotide of the compound to its target sequence. These mechanisms include, but are not limited to, interference with processing, inhibition of transport across the nuclear membrane, cleavage by endonucleases, formation of replicase complexes or the like.

The compounds described herein may be used in the treatment of infectious diseases. The target nucleic acid sequences include, but are not limited to, those genes of pathogenic viruses such as HIV, CMV, HSV, HCV, etc., as well as genes encoding host factors for these viruses or otherwise involved in disease development and/or progression.

In the treatment of cancer, the target nucleic acid sequences can be DNA or RNA associated with oncogenes or viruses with oncogenic properties, tumor suppressor genes, and related genes. Additionally, the compounds of the present invention may also target genes associated with drug resistance and their gene products.

The targeting process usually also includes determination of at least one target region, segment, or site within the target nucleic acid for the antisense interaction to occur such that the desired effect, e.g., modulation of expression, will result. Within the context of the present invention, the term “region” refers to a portion of the target nucleic acid having at least one identifiable structure, function, or characteristic. Within regions of target nucleic acids are segments. “Segments” refers to smaller or sub-portions of regions within a target nucleic acid. “Sites,” as used in the present invention, refer to positions within a target nucleic acid.

It is further contemplated that the compositions described herein are used in combination to hybridize to two different regions or segments within the same viral genome. For choosing a target, the considerations include: localization of the target in a region of the viral genome which is important for virus multiplication (target must be essential region). If possible a preferred target should be in a region which is conserved among different strains and genotypes of the virus (often this also indicates the functional significance of the sequences). The regions encoding highly conserved domains of proteins are good targets; also the regions containing overlapping functional elements (coding sequences overlapping with cis-active elements) are good targets.

It is further contemplated that the target site should have a nucleotide composition that enables construction of an oligonucleotide inhibitor with desirable nucleotide content and/or composition of modified nucleobases, and preferably the target does not contain strong secondary structural elements. Further, the sequence of the target should not overlap with that of essential host genes, especially host mRNAs. Additionally, the positions of modified nucleobases should not match a host sequence. The cluster of C or G nucleotides (three or more) should be avoided. Experiments have shown that target sites inside coding regions are better than those in non-coding regions and that in the case of RNA viruses, the positive strand is a better target than the negative strand. Due to the unique mechanism of nucleic acid destruction (e.g., by RNAse or DNAse complex) it is not necessary to target the modified oligonucleotide to the translation initiation sequence. This is in contrast to the case of morpholino oligonucleotides, which cannot initiate RNA degradation and are most (or exclusively) effective if targeted to the regions containing an initiation codon for translation. Such restriction does not exist for the currently described modified oligonucleotides.

For targeting two or more sites, each site should satisfy several of the criteria set out above. Sequences of the targets should be different and not complementary to each other to avoid aggregation of the oligonucleotide and the targets could represent different sequences from one and the same functional unit, for example from the same enzyme, or, from different units. In most cases the second option is the preferred to minimize the possibility of generation of resistant mutations. As used herein, the term “functional unit” refers to a polypeptide or polynucleotide sequence having a function in viral replication or gene expression, e.g., different replication factors, transcription factors, etc. Oligonucleotides that bind the same functional units bind different target sequences but in the same polypeptide or polynucleotide functional unit, e.g., within the HIV Tat protein. Oligonucleotides contemplated by the invention that bind different functional units bind to polypeptide or polynucleotide having different functions in viral replication or gene expression, e.g., HIV Tat and Rev genes or proteins. One of ordinary skill in the art can readily understand the meaning of a functional unit associated with viral replicaion or gene expression.

The translation initiation codon is typically 5′ AUG (in transcribed mRNA molecules; 5′ ATG in the corresponding DNA molecule), the translation initiation codon is also referred to as the “AUG codon,” the “start codon” or the “AUG start codon”. A minority of genes have a translation initiation codon having the RNA sequence 5′ GUG, 5′ UUG or 5′ CUG, and 5′ AUA, 5′ ACG and 5′ CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding Interleukin 18, regardless of the sequence(s) of such codons. It is also known in the art that a translation termination codon (or “stop codon”) of a gene may have one of three sequences, i.e., 5′ UAA, 5′ UAG and 5′ UGA (the corresponding DNA sequences are 5′ TAA, 5′ TAG and 5′ TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts.” It is also known that introns can be effectively targeted using antisense compounds targeted to, for example, DNA or pre-mRNA.

Alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants.” Consequently, mRNA variants are processed pre-mRNA variants and each unique pre-mRNA variant must always produce a unique mRNA variant as a result of splicing. These mRNA variants are also known as “alternative splice variants.” If no splicing of the pre-mRNA variant occurs then the pre-mRNA variant is identical to the mRNA variant.

Variants can be produced through the use of alternative signals to start or stop transcription and pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

Additionally, the majority of DNA genomic viruses as well as viruses with RNA genomes contain fragments of their genomes used for the initiation of the synthesis of new genomic strands. Their segments are designated as replication origins and are variable by structure, number (copies per genome) and by mode of action. For RNA viruses the replication origins typically contain sequences of both ends of RNA molecule. The sequence of replication origin is typically recognized by virus-encoded proteins, called origin recognition factors which are involved in initiation of replication and, in many cases, performing the replication process (in the latter case they are designated as “components of replicase complexes” or “viral replicases”). Viral replicases can consist almost completely from virus encoded proteins (as in case of HSV), from host- and viral subunits or be mostly encoded by host cells.

Some segments of viral sequences are recognized by virus, host or virus and host encoded factors. Such segments are replication origins, promoters, enhancers, terminators, splicing and polyadenylation signals, packaging sequences etc and they are commonly called “cis-acting elements.” The viral, host or viral-host protein complexes, interacting with these signals are called “trans-acting factors.” Cis-acting sequences may have specific primary and/or secondary structure required for their functioning and these elements can be located in different positions of viral genomes (including in coding sequences). Cis-acting elements may overlap with each other as well as with coding sequences encoding corresponding trans-acting factors.

Many DNA and almost all RNA viruses contain reading frames only in one strand of their genome (defined as “positive strand”). In case of RNA viruses with single stranded genomes the genome can be represented by the coding mRNA sequence (positive-strand RNA viruses) or by RNA complementary to that strand, packed into nucleocapsid protein (negative strand RNA viruses). During the replication in cells these viruses synthesize the RNA with opposite polarity defined as “antigenome” which is negative RNA for positive-strand RNA viruses and positive RNA for negative-strand RNA viruses. This strand may form a duplex with the genomic RNA (positive strand viruses) or not (negative strand RNA viruses). The duplex between positive- and negative-strands is defined as “replicating form” or “replicating intermediate” and, in the cells infected by positive-strand RNA viruses, it is bound to replicase proteins and cellular membranes.

The locations on the target nucleic acid to which the preferred antisense compounds hybridize are herein below referred to as “preferred target segments.” As used herein the term “preferred target segment” is defined as at least a 5-nucleobase portion of a target region to which an active antisense compound is targeted. While not wishing to be bound by theory, it is presently believed that these target segments represent portions of the target nucleic acid which are accessible for hybridization.

While the specific sequences of certain preferred target segments are set forth herein, one of skill in the art will recognize that these serve to illustrate and describe particular embodiments within the scope of the present invention. Additional preferred target segments may be identified by one having ordinary skill.

Target segments 5-150 nucleobases in length comprising a stretch of at least five consecutive nucleobases selected from within the illustrative preferred target segments are considered to be suitable for targeting as well.

Target segments can include DNA or RNA sequences that comprise at least the 5 consecutive nucleobases from the 5′-terminus of one of the illustrative preferred target segments (the remaining nucleobases being a consecutive stretch of the same DNA or RNA beginning immediately upstream of the 5′-terminus of the target segment and continuing until the DNA or RNA contains about 5 to about 150 nucleobases). Similarly preferred target segments are represented by DNA or RNA sequences that comprise at least the 5 consecutive nucleobases from the 3′-terminus of one of the illustrative preferred target segments (the remaining nucleobases being a consecutive stretch of the same DNA or RNA beginning immediately downstream of the 3′-terminus of the target segment and continuing until the DNA or RNA contains about 5 to about 150 nucleobases). One having skill in the art armed with the preferred target segments illustrated herein will be able, without undue experimentation, to identify further preferred target segments.

Once one or more target regions, segments or sites have been identified, antisense compounds are synthesized which are sufficiently complementary to the target, i.e., hybridize sufficiently well and with sufficient specificity, to give the desired effect, and incorporate at least one hydroxynucleobase or mercaptonucleobase which is complementary to a nucleobase in the sequence of the target regeion, segment, or site, and further incorporates a chelating moiety as described herein. The antisense compound is then contacted with an ion of a metal to allow for complexation of the ion to the compound. This resulting compound can then be used in antisense therapy mechanisms.

### Assays for Evaluation of the Antiviral Effect of Modified Oligonucleotides

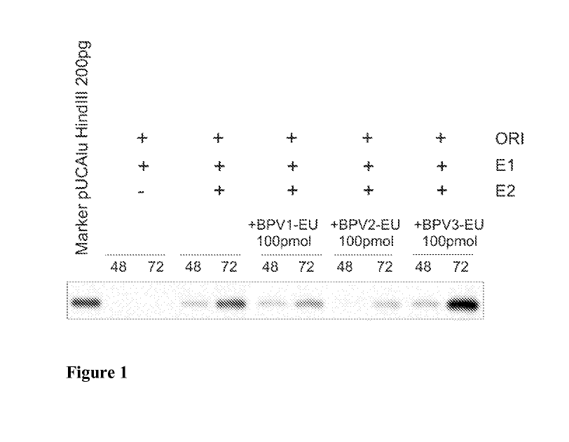

Alphaviruses are an example of a RNA virus that causes acute infection. Mosquitoes, birds and different mammals can all become infected by Semliki Forest virus (SFV). The genome of SFV is positive strand RNA approximately 11.5 kb in length. It contains two large open reading frames (ORFs)—one at the 5′ region and another at 3′ region (FIG. 1). The first ORF encodes non-structural proteins—the viral subunits of RNA replication system. The second ORF encodes virion proteins and is not needed for the RNA replication as such. Genomes of all known alphaviruses are organized in a similar manner. SFV replicates in the cytoplasm of infected cells; the replication process takes place on cellular membranes of endosomal and lysosomal origin. Typically the infection in mammalian cells leads to the almost complete shutdown of cellular RNA and protein synthesis and results in death of the infected cell at 12-24 h post infection. Thus, SFV is generally a highly cytotoxic and rapid virus.

The expression of SFV proteins can be monitored by use of SFV genomes with marker genes (Renilla Luciferase, Rluc) inserted in structural or nonstructural regions; the expression level of marker in infected cells is proportional to the copy number of viral RNAs (genomic RNAs if the marker is inserted in a non-structural region and subgenomic RNAs if the marker is inserted in a structural region) in infected cells (Kiiver et al., 2008). Thus, the monitoring of the Rluc activity by using EnduRen or any other appropriate substrate (Promega) provides adequate information about the viral replication (accumulation of viral RNAs and proteins) in infected cells.

Hepatitis C virus is an example of a RNA virus that causes chronic infection. HCV is non-cytopathic member of the family Flaviviridae. It has positive sense RNA genome about 9.6 kb in length. HCV genome lacks 5′ cap, but instead it carries an internal ribosomal entry site (IRES) within the 5′-UTR. HCV genome encodes for a single polyprotein, which becomes proteolytically cleaved into 10 individual proteins: four structural proteins and six non-structural proteins. Five of these non-structural proteins are required for replication of the HCV genome. The replication occurs in the cytoplasm of infected cells, where viral proteins and RNA together with cellular factors form a “membranous web” which serves as scaffold for the genome in replication complex. HCV replication is asymmetric (positive strands are more abundant). HCV virions are assembled on ER membranes and exit the cell through the secretory pathway. HCV does not induce general shutdown of host cell metabolism or cell death; instead it induces highly specific shutdown of the cellular anti-viral mechanisms.

The replication of HCV can be monitored by the use of a surrogate replicon model involving a transient expression system based on highly susceptible cell lines and HCV 1b replicons containing luciferase marker gene (replication-deficient genomes are used as negative controls).

The initial stages of HCV infection are difficult to reproduce in cell culture. Only limited HCV variants are capable to infect cultivated cells (JFH1 from genotype 2 and few recombinants based on the JFH1); so far it is not reported for the medically most important 1b genotype. The initial stages of infection of HCV 1b can be modeled, however, by using hybrid viruses containing a replicase part of non-cytotoxic mutant of SFV (Tamm et al., 1: J Gen Virol. 89:676-86, 2008) with inserted Rluc reporter and fragments of HCV replicase region cloned under the control of subgenomic promoter of SFV. These hybrid genomes can be packed into virus like particles with SFV structural proteins and used for infection of huh7 cells treated with anti-HCV and/or anti SFV compounds.

HIV-1 as an example of a pathogenic retrovirus. HIV-1 belongs to genus Lentivirus, family Retroviridae. The genome organization of HIV-1 is more complex than that of simple retroviruses. Genomic RNA of HIV is about 9 kb long and contains several genes. Some of these genes are expressed directly from the genome-size RNA, others are expressed by well-regulated alternative splicing. The gene expression of HIV is regulated by both viral and host factors. The best known viral regulators are Tat (transcriptional activator) and Rev (regulator of splicing and nuclear export). These proteins are expressed from the spliced RNA transcripts and their expression activates the subsequent transcription (Tat activity) and changes splicing pattern (Rev activity).

Tat-mediated activation of the HIV promoter is a model system which can be used for evaluation of antiviral compounds. When HIV provirus is integrated in the host genome, activation requires the interaction of HIV-encoded Tat-protein and Tar-element in viral mRNA. Without Tat protein, the transcription from the HIV promoter is very inactive and inclusion of Tat activates the promoter 300-fold. As a consequence, no active HIV gene expression can take place in the absence of Tat and Tar elements. The system for analysis is based on the Tat-activation of Tar-containing HIV promoter and contains the luciferase marker gene cloned under the control of HIV promoter (LTR) and this construct has been used to construct a stable Jurkat cell line. In these cells the expression of the luciferase marker takes place at a very low basal level. The expression can be activated by use of the Tat gene from B-clade virus HAN-2, delivered to these reporter cells by transfection. This process can be repressed by antiviral oligonucleotides.

Papillomaviruses as example of viruses with DNA genomes. Papillomaviruses are a group of small non-enveloped viruses with circular double-stranded DNA genomes. The replication cycle of papillomaviruses correlates strictly with development of epithelial tissue; virus maturates only in mature epithelial cells. In cells not developed to terminal stage, the papillomavirus genome replicates as a high copy (50-400 copies) per cell extrachromosomal plasmid. The DNA genome of papillomavirus is approximately 8 kbp long and encodes 10-12 proteins and replicates in the nucleus of the infected cell. The replication depends from two virus encoded proteins, E1 and E2. E2 is also involved in the regulation of viral gene expression.

The effects of the modified oligonucleotides on papillomavirus replication can be analyzed by monitoring the E2-mediated activation of gene expression. The reporter gene (firefly luciferase, Luc) was cloned under the control of the E2-activated promoter. By co-transfection of this reporter plasmid with E2 encoding plasmid the activation effect (and its modulation by antiviral oligonucleotides) can be measured.

Analysis of BPV replication in cells is another model for analysis of the effects of antiviral compounds. The viral copy number can be analyzed by quantitative PCR or Southern blotting.

### Formulations

The compounds of the invention may also be admixed, encapsulated, conjugated or otherwise associated with other molecules, molecule structures or mixtures of compounds, as for example, liposomes, carriers, diluents, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE ((S acetyl-2-thioethyl) phosphate) derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions useful in the present invention include, but are not limited to, solutions, emulsions, foams and liposome-, micelle-, or nanoparticle-containing formulations. The pharmaceutical compositions and formulations of the present invention may comprise one or more penetration enhancers, carriers, excipients, diluents, or other active or inactive ingredients.

Emulsions are typically heterogenous systems of one liquid dispersed in another in the form of droplets usually exceeding 0.1 μm in diameter. Emulsions may contain additional components in addition to the dispersed phases and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations useful in the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH sensitive or negatively charged are believed to entrap DNA rather than complex with it. Both cationic and noncationic liposomes have been used to deliver DNA to cells, and can be used to deliver compounds of the invention.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety. Liposomes containing the compositions described herein may also be conjugated or otherwise linked to a cell-penetrating peptide, such that the liposome is targeted into tissue and across a cell membrane. Cell-penetrating peptides include the HIV tat peptide, antibodies or binding fragments thereof, biotin, and other compositions known in art (Sawant et al., Bioconjug Chem. 17:943-9 (2006); Sapra et al., Curr Drug Deliv. 2:369-81 (2005)).

Additional nanoparticulate pharmaceutical carriers such as micelles and nanoparticles are contemplated for delivery of the compositions desecribed herein in vivo. Techniques for synthesizing and administering such nanopharmaceuticals are known in the art and described for example in Torchilin, V P., (Biopolymers. Mar. 31, 2008), Torchilin, V P, (Biochem. Soc. Trans. 35:816-820 (2007)), and Sawant et al. (Bioconjug Chem. 17:943-9 (2006)).

The pharmaceutical formulations and compositions of the present invention may also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to affect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the compounds of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, compounds of the invention may be encapsulated within liposomes or may form complexes thereto, in particular to cationic liposomes. Alternatively, compounds may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which compounds of the invention are administered in conjunction with one or more penetration enhancers, surfactants, and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Compounds of the invention may be delivered orally, in granular form including sprayed dried particles, or complexed to form micro or nanoparticles. Complexing agents and their uses are further described in U.S. Pat. No. 6,287,860, which is incorporated herein in its entirety. Oral formulations and their preparation are described in detail in U.S. application Ser. Nos. 09/108,673, 09/315,298, and 10/071,822, each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more compounds of the invention and one or more chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of antisense compounds and other non-antisense drugs are also within the scope of this invention. Two or more combined compounds may be used together or sequentially.

In another related embodiment, compositions of the invention may contain one or more antisense compounds, particularly oligonucleotides, targeted to a first nucleic acid target and one or more additional antisense compounds targeted to a second nucleic acid target. Alternatively, compositions of the invention may contain two or more antisense compounds targeted to different regions of the same nucleic acid target. Numerous examples of antisense compounds are known in the art. Two or more combined compounds may be used together or sequentially.

### Dosing

The formulation of therapeutic compositions and their subsequent administration (dosing) is believed to be within the skill of those in the art, and determined, e.g., by dose-response, toxicity, and pharmacokinetic studies. Dosing is dependent on severity and responsiveness of the disease state to be treated, with the course of treatment lasting from several days to several months, or until a cure is effected or a diminution of the disease state is achieved. Dosing may continue indefinitely for chronic disease states or conditions for which diminution but no cure can be achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

### Kits and Diagnostic Tools

The compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480:17-24; Celis, et al., FEBS Lett., 2000, 480:2-16), SAGE (serial analysis of gene expression)(Madden, et al., Drug Discov. Today, 2000, 5:415-425), READS (restriction enzyme amplification of digested cDNAs) (Prashar and Weissman, Methods Enzymol., 1999, 303:258-72), TOGA (total gene expression analysis) (Sutcliffe, et al., Proc. Natl. Acad. Sci. U.S.A., 2000, 97:1976-81), protein arrays and proteomics (Celis, et al., FEBS Lett., 2000, 480:2-16; Jungblut, et al., Electrophoresis, 1999, 20:2100-10), expressed sequence tag (EST) sequencing (Celis, et al., FEBS Lett., 2000, 480:2-16; Larsson, et al., J. Biotechnol., 2000, 80:143-57), subtractive RNA fingerprinting (SuRF) (Fuchs, et al., Anal. Biochem., 2000, 286:91-98; Larson, et al., Cytometry, 2000, 41:203-208), subtractive cloning, differential display (DD) (Jurecic and Belmont, Curr. Opin. Microbiol., 2000, 3:316-21), comparative genomic hybridization (Carulli, et al., J. Cell Biochem. Suppl., 1998, 31:286-96), FISH (fluorescent in situ hybridization) techniques (Going and Gusterson, Eur. J. Cancer, 1999, 35:1895-904) and mass spectrometry methods (To, Comb. Chem. High Throughput Screen, 2000, 3:235-41).

The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutics, a subject, preferably a human, suspected of having a disease or disorder which can be treated by modulating the expression of a target nucleic acid is treated by administering antisense compounds in accordance with this invention. For example, in one non-limiting embodiment, the methods comprise the step of administering to the subject in need of treatment, a therapeutically effective amount of an antisense compound. The compounds of the invention can be utilized in pharmaceutical compositions by adding an effective amount of a compound to a suitable pharmaceutically acceptable diluent or carrier. Use of the compounds and methods of the invention may also be useful prophylactically. For example, the compositions are useful to treat uninfected cells to decrease the viral gene expression and replication in these cells.

Additional aspects and details of the disclosure will be apparent from the following examples, which are intended to be illustrative rather than limiting.

### EXAMPLES

### Example 1

### Use of Modified Oligonucleotide for Inhibition of the Semliki Forest Virus

To determine the effects of modified oligonucleotides on viral gene replication, antisense oligonucleotides against Semliki Forest virus (SFV), a rapid (acute) positive strand RNA virus from genus Alphavirus family Togaviridae, were generated and inhibition of viral genome replication measured.

A list of oligonucleotides and targets inside of the SFV viral genome is set out in Table 1.

TABLE 1

| | Sequence of compound | Presence | Target | Orientation |
|---|---|---|---|---|
| | (5′-3′), 5′ end had | of organic | region of | with respect |
| Code | amino groups | RNAse | SFV genome | to target |
| A6 | TTA ATC TCT CXC GTA GCG | No | 5′ UTR | antisense |
| | GAG XT | | | |
| | (SEQ ID NO. 1) | | | |
| A6-N | TTA ATC TCT CXC GTA GCG | Yes | 5′ UTR | antisense |
| | GAG XT | | | |
| | (SEQ ID NO. 1) | | | |
| A7 | ATX TTX TCG TCG CCG ATG | No | nsP4 | antisense |
| | AAG XC | | | |
| | (SEQ ID NO. 2) | | | |
| A7-N | ATX TTX TCG TCG CCG ATG | Yes | nsP4 | antisense |
| | AAG XC | | | |
| | (SEQ ID NO. 2) | | | |
| A8 | GCC TTC ATC GXC GAC XAC | No | nsP4 | sense |
| | AAC AT | | | |
| | (SEQ ID NO. 3) | | | |
| A8-N | GCC TTC ATC GXC GAC XAC | Yes | nsP4 | sense |
| | AAC AT | | | |
| | (SEQ ID NO. 3) | | | |

X - modified base, 8-oxo-dG
nsP4 - conserved part of the coding region for viral RNA polymerase, the non-structural protein 4

BHK-21 (Baby hamster kidney) cells were grown to confluency on a 35 mm diameter dish, the lipofectamine RNAiMAX (Invitrogen) was used to transfect cells with modified oligonucleotides with or without a metal nuclease (Eu). Transfection was done either at the moment of infection or prior to infection. Cells were transfected with 30 picomol of oligonucleotides (irrelevant oligonucleotides with or without organic nuclease were used as controls) and infected at multiplicity of infection (moi) 0.05 with reporter virus SFV4-stRLuc (self-replicating SFV vector with Renilla luciferase inserted into the structural region of the SFV genome). Some conditions such as moi, amount of transfection reagent and the time interval between transfection and infection were varied in different experiments. EnduRen Live Cell Substrate (Promega) was used to detect the R-Luc activity, measurements were done at 1.5, 2, 3, 4, 5, and 8 hours post infection in most experiments (later time-points were used in some experiments as well). Results from the experiment are shown in Table 2.

TABLE 2

| | Reduction of the virus expressed Rluc activity |
|---|---|
| | (folds) in cells treated with indicated agent |
| Agent | with respect to cells treated with control agent |
| A6 | 1.14 (control agent did not containe nuclease) |
| A6-N | 1.18 (control agent contained organic nuclease) |
| A7 | 1.66 (control agent did not containe nuclease) |
| A7-N | 5.61 (control agent contained organic nuclease) |
| A8 | 0.99 (control agent did not containe nuclease) |
| A8-N | 0.75 (control agent contained organic nuclease) |

Comparison of three targets for inhibition of SFV replication revealed that oligonucleotide A8 (sense type oligos, target is antisense RNA) have no effect on viral replication, while A6 oligonucleotides (antisense, target is 5′UTR of SFV) have a moderate effect and A7 oligonucleotides (antisense, target is coding region for nsP4) have a significant inhibitory effect which is magnified 3.4 times when complexed with organic nuclease (Eu).

### Example 2

### Use of Modified Oligonucleotide for Inhibition of the Hepatitis C Virus

To determine the effects of modified oligonucleotides on viral gene replication, antisense oligonucleotides against Hepatitis C virus (HCV), a slow (chronic) positive strand RNA virus from genus Hepacivirus, family Flaviviridae, were generated and inhibition of the viral genome replication measured.

A list of oligonucleotides and targets inside the HCV viral genome is given in Table 3.

TABLE 3

| | | Presence | Target | Orientation |
|---|---|---|---|---|
| | Sequence of compound | of | region of | with |
| | (5′-3′), 5′ end had | organic | HCV | respect to |
| Code | amino groups | RNAse | genome | target |
| B4 | CTTTYACAGATAACGAYAAGGTC | No | NS5B | antisense |
| | (SEQ ID NO. 4) | | | |
| B4-N | CTTTYACAGATAACGAYAAGGTC | Yes | NS5B | antisense |
| | (SEQ ID NO. 4) | | | |
| B6 | CTTTCACAXATAACGACAAXGTC | No | NS5B | antisense |
| | (SEQ ID NO. 4) | | | |
| B6-N | CTTTCACAXATAACGACAAXGTC | Yes | NS5B | antisense |
| | (SEQ ID NO. 4) | | | |

X - modified base, 8-oxo-dG; Y- modified base, 5-OH-dC
NS5B - conserved part of the coding region for viral RNA polymerase, the non-structural protein 5B

Analysis of HCV replication and the effects of inhibitors on this process were carried out using HCV replicons contain Luc-marker gene and were delivered into Lunet cells as RNA transcripts. HCV replicons (genotype 1b) with no selection marker, containing Luc-marker under control of HCV IRES and HCV nonstructural region (NS3-NS5B) with native 3′ UTR of virus were used for the assay. cDNAs from corresponding constructs were transcribed in vitro and transfected to huh7/Lunet cells by electroporation together with a modified oligonucleotide compound. The HCV initiates transient replication where the expression of luciferase is proportional to the copy number of HCV genomes. The luciferase activity was measured from 4-96 h post-transfection at 4 hours, 24 hours, 48 hours, 72 hours and 96 hours (in some experiments additional timepoints were also used). The cell culture was split several times during the experiment and the luc-expression level was normalized to the total protein in samples.

The expression of luciferase is proportional to the copy number of HCV genomes and was measured at the timepoints set out above. Modified nucleotide-based inhibitors (100 picomol) with or without nuclease complex (Eu) were added together with viral RNA (co-electroporation). Results from the experiment are shown in Table 4.

TABLE 4

| | Reduction of the virus expressed luciferase activity |
|---|---|
| | (folds) in cells treated with indicated agent |
| Agent | compared to cells treated with control agent |
| B4 | 2.27 (control agent did not containe nuclease) |
| B4-N | 2.45 (control agent contained organic nuclease) |
| B6 | 1.71 (control agent did not containe nuclease) |
| B6.N | 2.62 (control agent contained organic nuclease) |

These results show that inhibition of HCV replication by use of the modified oligonucleotides was observed with oligonucleotides having either 8-oxo-dG; or 5-OH-dC. Modified oligonucleotides complexed with the organic nuclease contributed to antiviral activity and demonstrated an antiviral effect at 10-fold lower concentrations than oligos lacking the nuclease complex.

### Example 3

### Use of Modified Oligonucleotide for Inhibition of Tat-Activated Gene Expression of Human Immunodeficiency Virus Type 1 (HIV-1)

To determine the effects of modified oligonucleotides on viral gene replication, antisense oligonucleotides against HIV-1, genus Lentivirus family Retroviridae, were generated and inhibition of viral genome replication measured.

A list of oligonucleotides and targets inside the HIV viral genome are given in Table 5.

TABLE 5

| | | Presence | Target | Orientation |
|---|---|---|---|---|
| | Sequence of compound | of | region of | with |
| | (5′-3′), 5′ end had | organic | HIV | respect to |
| Code | amino groups | RNAse | genome | target |
| A11 | CTA XCC AGA GAG CTC CCA | No | Tar | antisense |
| | GXC TC | | | |
| | (SEQ ID NO. 6) | | | |
| A11- | CTA XCC AGA GAG CTC CCA | Yes | Tar | antisense |
| N | GXC TC | | | |
| | (SEQ ID NO. 6) | | | |
| A12 | TTT CTT XTG AAA GAA ACT | No | Tat | antisense |
| | TGX CA | | | |
| | (SEQ ID NO. 7) | | | |
| A12- | TTT CTT XTG AAA GAA ACT | Yes | Tat | antisense |
| N | TGX CA | | | |
| | (SEQ ID NO. 7) | | | |
| A13 | CTA GCC AGA GAG CTC CCA | No | Tar | antisense |
| | GGC TC | | | |
| | (SEQ ID NO. 8) | | | |
| A14 | TTT CTT GTG AAA GAA ACT | No | Tat | antisense |
| | TGG CA | | | |
| | (SEQ ID NO. 9) | | | |

X - modified base, 8-oxo-dG
Tar - conserved hairpin loop in 5′ region of HIV RNA; Tat- HIV encoded protein, binds to Tar-element and activates the transcription of viral mRNAs and genomes from integrated provirus.

Jurkat cell lines with HIV promoter—luciferase reporter constructs were used for these experiments. The activator of expression (Tat expressing plasmid, 5 ng) was delivered into cells together with oligonucleotide inhibitors (100 picomols). The effect(s) of inhibitors were calculated by comparison of the levels of measured luciferase activities (24 hours post-induction). Results from the experiment are shown in Table 6.

TABLE 6

| | Fold Reduction of the luc-activity in cells treated with indicated |
|---|---|
| Agent | agent compared to activities measured from untreated cells. |
| A11 | 0.89 |
| A11-N | 0.96 |
| A12 | 1.34 |
| A12-N | 2.09 |
| A13 | 0.92 |
| A14 | 0.84 |

The HIV-Tat sequence is an effective target for modified antisense oligonucleotides. The inhibitory effect was detected only when the oligonucleotide contained modified bases and was further increased by the presence of RNA nuclease activity.

### Example 4

Use of S-Modified Oligonucleotide for Inhibition of E2-Activated Gene Expression and Replication of Bovine Papillomavirus Type 1

Previous results have shown that oligonucleotides having modified bases containing either hydroxy or mercapto modification are more efficient in antisense inhibition of gene replication (See WO 2007/125173, incorporated herein by reference). To determine the effects of these modified oligonucleotides on viral gene replication, various modified oligonucleotides were assayed for their ability to inhibit replication of the bovine papilloma virus (BPV) E2 oncogene.

Based on pre-screening for potential targets by the use of non-modified oligonucleotides and siRNA, the sequence 5′ TGGAGACAGCATGCGAACGTTTA 3′ (SEQ ID NO. 10), located in the 5′ region of the mRNA encoding the E2 protein of BPV1 was selected as a target for modified oligonucleotide based inhibitors. Three inhibitors were constructed against these sequences, and all three compounds contained a nuclease complex with europium at their 5′ end as described in WO 2007/125173.

| (SEQ ID NO. 11) | |
|---|---|
| BPV1_Eu: | 5′ TAAACGTTCGCATGCTGTCTCCA 3′ |
| (SEQ ID NO: 12) | |
| BPV2_Eu: | 5′ TAAAXGTTCGCATGCTGTCTCCA 3′ |
| | where X = 5-SH-dC |
| (SEQ ID NO. 13) | |
| BPV3_EU: | 5′ TAAACXTTCGCATGCTGTCTCCA 3′ |
| | where X = 8-SH-dG |

Test of E2-mediated activation of luciferase expression in CHO cell line. CHO cells do not express the E2-transgene, and therefore, the co-transfection with E2-expressing plasmid was necessary to analyze oligonucleotide activity against the E2 gene. To avoid E2 overexpression, the amount of E2-expressing plasmid required for significant, but still dynamic, activation was established. The E2-independent expression of Renilla luciferase (Rluc) was used for normalization of the results. Based on previous dose response analysis, 1 ng of E2-expression plasmid was selected for the assays. Transfection of 1 ng of E2-expression plasmid caused 4 to 5-fold activation of marker expression (a 4 to 5-fold reduction of marker expression corresponds to complete absence of E2-mediated activation) and at that concentration a small change in E2 expression level resulted in significant changes of overall activation of marker expression.

One hundred picomoles of modified oligonucleotide inhibitors were delivered to CHO cells together with 1 ng E2-expressing plasmid and reporter plasmid and the inhibition of E2-dependent luciferase expression was observed. BPV1_EU caused a 1.6-fold reduction in reporter expression, while both BPV2_EU and BPV3_EU caused approximately 2-fold reduction in E2 activated gene expression. Based on the previously calculated calibration curve, the 1.6-fold reduction of Luc to Rluc ratio corresponds to a 2-fold decrease of E2 expression, and a 2-fold reduction of Luc to RLuc ratio corresponds to a 3.5-fold decrease of E2 expression.

Suppression of BPV replication using modified oligonucleotide inhibitors against E2 mRNA. BPV replication was measured in CHO cells co-transfected with plasmid containing BPV-1 minimal replication origin and expression plasmids for E1 and E2 proteins. Southern blotting was used to analyze the copy number of recombinant viral origins in transfected cells. In this assay all three anti E2-modified oligonucleotides with nuclease complexes (BPV1_EU, BPV2_EU and BPV3_EU) were analyzed. Results show that 100 picomol of BPV1_EU caused slight inhibition of replication at 72 hours post-transfection. The same amount of BPV2_EU caused increased inhibition which was detectable both at 48 and 72 hours post-infection. The same amount of BPV3_EU demonstrated little if any suppression of replication. Results are illustrated in FIG. 1.

These results show that antisense modified oligonucleotides with nuclease complexes are able to repress the E2-activated gene expression and E2-dependent replication of papilloma virus. Compared to the non-modified oligonucleotides targeted against the same site, the antisense modified oligonucleotides were more efficient inhibitors. Antisense modified oligonucleotides having a 5-SH-dC residue was a more efficient inhibitor of PV replication than antisense modified oligonucleotides with 8-5H-dG residue. In contrast, there was no observed difference between differently modified inhibitors in the ability to repress E2-activated transcription.

### Example 5

### Use of Mercaptonucleobases Increases the Antiviral Efficiency and Reduces the Concentration of Modified Oligonucleotides Necessary for Inhibition

The evidence that oligonucleotides with modified hydroxybases reduce replication of hepatitis C virus (HCV) in a transient expression model was described in Example 2. To determine if oligos having mercaptonucleobases have the same increased antiviral effect, the inhibitory effect of 100 picomols of oligonucleotide B4_N (containing 2 5-OH-dC bases) or 100 picomol of agent B6-N (containing 2 8-oxo-dG bases) (Table 7) is measured in a replication assay as described previously.

Results show that the mercapto-oligonucleotides caused a reduction in HCV replication. When similar experiments were repeated with oligonucleotides containing the same nucleotide sequence, except that the 5-OH-dC bases of B4-N were substituted with 5-5H-dC bases and 8-oxo-dG bases of B6-N were substituted with 8-5H-dG bases (Table 7) the inhibitory effects caused by B4S-N and B6S-N were detectable at 40 picomol amounts, demonstrating that substitution of the hydroxybases with mercaptobases reduced the amount of inhibitor required to achieve the inhibition of HCV replication in a transient system by approximately 2.5 fold.

TABLE 7

| | | | Effect on the | |
|---|---|---|---|---|
| | | | HCV | Reduction |
| | | | replication in | of |
| | | | transient | replication |
| | | | system | of hybrid |
| | | | detected at | virus with |
| | | | (minimal | HCV target |
| | | | active | site (folds) |
| | | Modified | amount) in | at 30 |
| Agent | Sequence, 5′-3′ | base | picomols | picomol |
| B4-N | CTTTYACAGATAACGAYAAGGTC | 5-OH-dC | 100 | Not |
| | (SEQ ID NO. 4) | | | detected |
| B4S- | CTTTZACAGATAACGAZAAGGTC | 5-SH-dC | 40 | 3.4 |
| N | (SEQ ID NO. 14) | | | |
| B6-N | CTTTCACAXATAACGACAAXGTC | 8-oxo- | 100 | Not |
| | (SEQ ID NO. 5) | dG | | detected |
| B6S- | CTTTCACAWATAACGACAAWGTC | 8-SH-dG | 40 | 1.5 |
| N | (SEQ ID NO. 15) | | | |

When oligonucleotides B4S-N and B6S-N were used for inhibition of the replication of the hybrid virus containing a replicase part of the non-cytotoxic SFV mutant with inserted marker and HCV fragment with respective target site (NS5A-5B region), the strong inhibition of the hybrid virus replication was observed when only 30 picomols of inhibitors was used. The overall reduction of hybrid virus replication was 1.5 fold for B6S-N and 3.4 fold for B4S-N.

These results demonstrate that use of mercaptobases instead of hydroxybases increases the antiviral efficiency and reduces the active concentration of modified oligonucleotides necessary for inhibition of HCV transient replication. The enhanced effect may result from enhanced binding of such modified oligonucleotides to the target, from increased affinity of such modified oligonucleotides to the membranes (where viruses replicate), from an increased ability of these modified oligonucleotides, on their own or in combination with transfection reagents, to penetrate cell membranes, from the increased stability of the oligonucleotides with these modifications in cellular environment and/or outside of the cell, or from any combination of these mechanisms.

### Example 6

### Increased Number of Modified 8-oxo-dC Nucleobases Reduces the Amount of Inhibitor Needed to Inhibit Virus Replication

To determine the effects of the location of the modification and the number/percent of modified nucleobases in the anti-viral activity of modified oligonucleotides, constructs having varied numbers of modified 8′ or 5′ hydroxynucleobases were generated and used in antiviral inhibition assays.

The effect of the increase of the number of modified bases per oligonucleotide is illustrated by the example of inhibition of HCV replication by modified oligonucleotide containing increasing number of modified 8-oxo-dG residues. The sequence of the inhibitor was antisense to the region of NS5B of HCV 1b genotype and had the sequence: 5′ GGGCGGGTCCCCAGGGGGGGCAG 3′ (SEQ ID NO. 16) where zero, one, five, ten or thirteen G-residues were substituted with 8-oxo-dG residues. These five compounds were used for inhibition of the replication of the hybrid virus, containing a replicase part of the non-cytotoxic SFV mutant with an inserted marker and HCV fragment with the respective target site (NS5AB region). Cells were treated by modified oligonucleotides 24 hours prior to infection with hybrid virus particles, 30 picomols of the inhibitors were used per 1 million of huh7 cells. The RLuc activities, corresponding to the copy number of recombinant virus genomes, were measured 24 hours post-infection. The results are provided in Table 8.

TABLE 8

| | | % of 8- | % of 8- | | |
|---|---|---|---|---|---|
| | Number | oxo-dG | oxo-dG | | |
| | of 8-oxo- | residues | residues | Sequence of the | Fold |
| | dG | (from | (from | inhibitior, 5′ to | Reduction |
| Name of | residues in | total G | total | 3′ X indicates | of RLuc |
| compound | compound | residues) | residues) | the modified base | activity |
| HMO_1 | 0 | 0 | 0 | GGGCGGGTCCCCAGGGGGGGCAG | None |
| | | | | (SEQ ID NO. 16) | |
| HMO_2 | 1 | 7.7 | 4.3 | GGGCGXGTCCCCAGGGGGGGCAG | None |
| | | | | (SEQ ID NO. 17) | |
| HMO_3 | 5 | 38.5 | 21.7 | GGXCXXGTCCCCAGGGXXGGCAG | None to 2 |
| | | | | (SEQ ID NO. 18) | |
| HMO_4 | 10 | 76.9 | 43.5 | GXXCXXXTCCCCAXXXXGGXCAG | 1.4 to 4 |
| | | | | (SEQ ID NO. 19) | |
| HMO_5 | 13 | 92.9 | 56.5 | XXXCXXXTCCCCAXXXXXXXCAG | 2.7 to 4 |
| | | | | (SEQ ID NO. 20) | |

HMO_{—}1 used in higher amounts demonstrated inhibition similar to the first three modified oligonucleotides. These results show that the increase in the number of 8′-oxo-dG bases in the oligo inhibitor results in increased antiviral effects detected at 30 picomols per million treated cells.

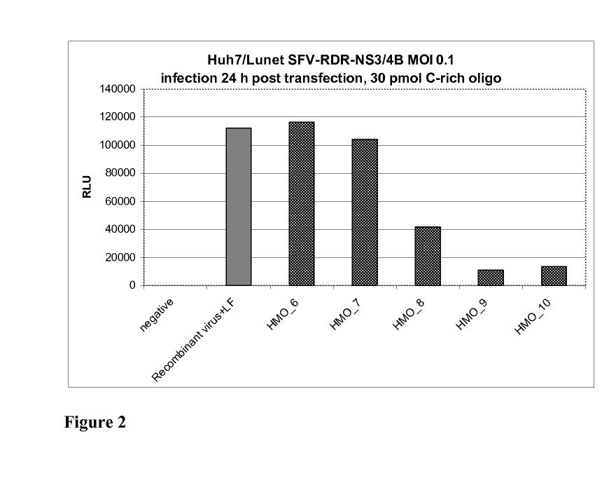

Inhibition of HCV replication using modified oligonucleotides containing an increasing number of modified 5-OH-dC residues was performed using inhibitors antisense to the region of NS4B of HCV 1b genotype and having the sequence: 5′ CTGCACAGCCCCCTCCCCTGGGC 3′ (SEQ ID NO. 21), where zero, one, five, ten or twelve C-residues were substituted with 5′-OH-dC residues. These five compounds were used for inhibition of the replication of the hybrid virus, containing a replicase part of the non-cytotoxic SFV mutant with inserted marker and HCV fragment with respective target site (NS3-4B region) as described above. Results are provided in Table 9 and FIG. 2.

TABLE 9

| | | % of 5- | % of 5- | | |
|---|---|---|---|---|---|
| | | OH-dC | OH-dC | | |
| | 5-OH-dC | residues | residues | Sequence of the | Fold |
| | residues | (from | (from | inhibitor, 5′ to | Reduction |
| Name of | in | total C | total | 3′ Y indicates | of RLuc |
| compound | compound | residues) | residues) | the modified base | activity |
| HMO_6 | 0 | 0 | 0 | CTGCACAGCCCCCTCCCCTGGGC | 0.96 |
| | | | | (SEQ ID NO. 21) | |
| HMO_7 | 1 | 7.7 | 4.3 | CTGYACAGCCCCCTCCCCTGGGC | 1.07 |
| | | | | (SEQ ID NO. 22) | |
| HMO_8 | 5 | 38.5 | 21.7 | CTGYACAGYYCCCTCYYCTGGG | 1.3-2.7 |
| | | | | C | |
| | | | | (SEQ ID NO. 23) | |
| HMO_9 | 10 | 76.9 | 43.5 | CTGYACAGYYYYYTYYYYTGGG | 5.6-9.8 |
| | | | | C | |
| | | | | (SEQ ID NO. 24) | |
| HMO_10 | 12 | 92.3 | 52.2 | YTGYAYAGYYYYYTYYYYTGGG | 8.1-10 |
| | | | | C | |
| | | | | (SEQ ID NO. 25) | |

These results showed that compared to oligonucleotides with a single 5-OH-dC base the oligonucleotide having five 5-OH-dC bases increased the inhibitory effect 2.5 fold and the oligonucleotide having ten 5-OH-dC bases increased inhibition more than 9 fold compared to the single base replacement.

### Example 7

### Nuclease Complex Increases Antiviral Efficiency of Oligonucleotides with High Content of Modified 8-oxo-dG, 5-oxo-dC or 6-OH-dU Bases

In order to assess the effect of the presence of a nuclease complex in the modified oligonucleotide containing a high number of modified bases per oligonucleotide, inhibition of HCV replication by modified oligonucleotides containing zero, five or ten modified 5-OH-dC residues was analyzed.

The sequence of the inhibitor was antisense to the region of NS4B of HCV 1b genotype and had sequence: 5′ CTGCACAGCCCCCTCCCCTGGGC 3′(SEQ ID NO. 21) where zero, five or ten C-residues were substituted with 5′-OH-dC residues; the oligonucleotides either had nuclease complex or not. These six compounds were used for inhibition of the replication of the hybrid virus, containing replicase part of non-cytotoxic SFV mutant with inserted marker and HCV fragment with respective target site (NS3-4B region). Cells were treated by modified oligonucleotides 24 hours prior to infection with hybrid virus particles, 30 picomols of these inhibitors was used per 1 million of huh7 cells. The RLuc activities, corresponding to the copy number of recombinant virus genomes, measured 24 hours post-infection, are provided in Table 10.

TABLE 10

| Number of | % of 5-OH-dC | Presence | Reduction of the RLuc activity (replication of hybrid |
|---|---|---|---|
| 5-OH-dC | residues | of the | virus) in inhibitor-treated cells compared to the |
| residues in | (from total | nuclease | untreated control cells. Factor of enhancement |
| compound | C residues) | complex | by nuclease is given in parentheses. |
| 0 | 0 | No | 0.96 |
| 0 | 0 | Yes | 1.3 (1.38) |
| 5 | 38.5 | No | 2.7 |
| 5 | 38.5 | Yes | 4.3 (1.59) |
| 10 | 76.9 | No | 9.8 |
| 10 | 76.9 | Yes | 29.9 (3.1) |

These results show that addition of the nuclease complex increased the antiviral effect of oligonucleotides with five or ten 5-OH-dC bases. The enhancement of inhibition is dependent on the efficiency of the inhibitor without the nuclease; the more active the compound itself, the more additive effect is provided by the nuclease.

The same principle is demonstrated for oligonucleotides with five or ten modified 8-oxo-dG residues and for oligonucleotides with nine 6-OH-dU residues.

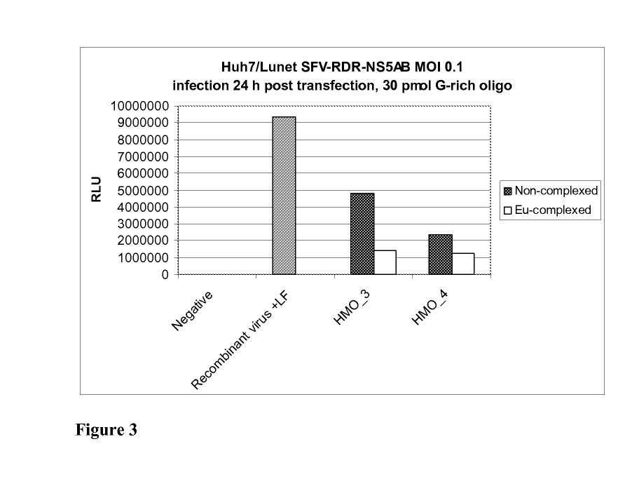

In an additional assay, an inhibitor antisense to the region of NS5B of HCV 1b genotype having the sequence: 5′ GGGCGGGTCCCCAGGGGGGGCAG 3′ (SEQ ID NO. 16) where five or ten G-residues were substituted with 8′-oxo-dG residues, with or without nuclease complex was tested. These six compounds were used for inhibition of the replication of the hybrid virus, containing a replicase part of the non-cytotoxic SFV mutant with inserted marker, and an HCV fragment with respective target site (NS5AB region). Cells were treated by modified oligonucleotides 24 hours prior to infection with hybrid virus particles, 30 picomols of these inhibitors was used per 1 million of huh7 cells. The RLuc activities, corresponding to the copy number of recombinant virus genomes, measured 24 hours post-infection, are provided in FIG. 3.

These results show that the addition of the nuclease complex increases the antiviral effect of oligonucleotides with five or ten 8-oxo-dG residues, thus the results can be logically extended to the oligonucleotide containing any number of any hydroxyl-modified nucleobases.

The experiments above may be repeated using an oligonucleotide having nine T-residues substituted by 6-OH-dC residues. It is expected that oligos having these substitutions have similar inhibitory activity as the oligos having the 8-oxo-dG residues.

### Example 8

### Nuclease Complex Reduces the Effective Concentration of the Modified Nucleotide with High Content of Hydroxy-Modified Nucleobases

To determine the effect of the nuclease complex in the oligonucleotides containing a high number of the number of modified bases per oligonucleotide, on the effective concentration 50 of the compound, inhibition of HCV replication by modified oligonucleotide containing ten 5-OH-dC residues with or without nuclease complexes was examined.

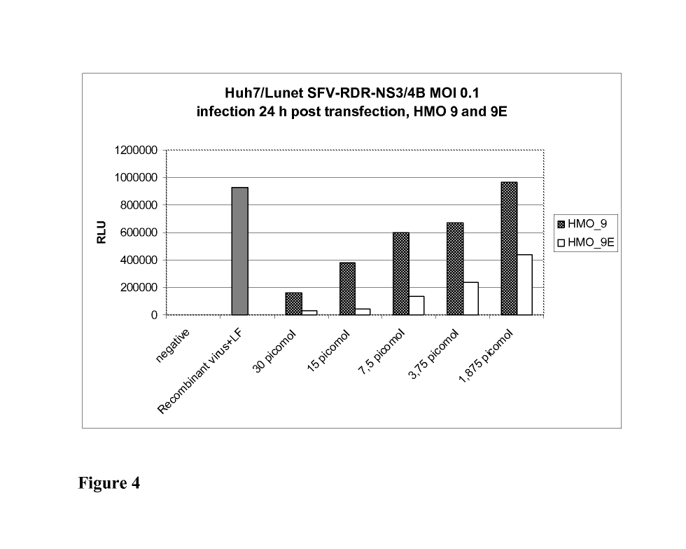

An inhibitor antisense to the region of NS4B of HCV 1b genotype and having the sequence: 5′ CTGCACAGCCCCCTCCCCTGGGC 3′ (SEQ ID NO. 21), where ten C-residues were substituted with 5′-OH-dC residues, was used in the inhibition assay. The oligonucleotides with or without nuclease complex were also examined. Serial dilutions of these compounds were used for inhibition of the replication of the hybrid virus, containing a replicase part of the non-cytotoxic SFV mutant with inserted marker, and an HCV fragment with respective target site (NS3-4B region). Cells were treated by modified oligonucleotides 24 hours prior to infection with hybrid virus particles, 30, 15, 7.5, 3.75 or 1.875 picomols of these inhibitors per 1 million of huh7 cells. The RLuc activities, corresponding to the copy number of recombinant virus genomes, measured 24 hours post-infection, are provided in Table 11 and in FIG. 4.

TABLE 11

| | |
|---|---|
| | Reduction of the RLuc activity (replication |
| Amount of | of hybrid virus) in inhibitor-treated cells |
| inhibitor | compared to the untreated control cells. |

| (picomoles) | No nuclease | With nuclease |
|---|---|---|
| 30 | 5.7 | 27.2 |
| 15 | 2.4 | 19.4 |
| 7.5 | 1.6 | 6.9 |
| 3.75 | 1.4 | 3.8 |
| 1.875 | 1 | 2.1 |

The cells were treated with two-fold dilutions of antiviral oligonucleotides and their anti-viral efficiency was analyzed by reduction of the replication of recombinant virus containing the corresponding target site FIG. 4) Inhibition of replication of recombinant virus was monitored by measuring Rluc activity expressed from genomic RNA of the virus.

These results show that the addition of the nuclease complex increases the antiviral effect of an inhibitory oligonucleotide having ten modified bases and reduces the amount of inhibitor required to achieve 50% of reduction of virus replication from approximately 10 picomols to less than 1.875 picomols. It is expected that the same principle applies for oligonucleotides having any modified nucleobases or any combination of modified nucleobases.

To determine the effect of an oligonucleotide having multiple 6-OH-dU bases per oligonucleotide on inhibition of HCV replication, an oligonucleotide containing nine 6-OH-dU residues is used in an inhibition assay as described above. An inhibitor antisense to the region of NS3 of HCV 1b genotype and having the sequence: 5′ AGTTGTCTCCTGCCTGCTTAGTC 3′ (SEQ ID NO. 26) where zero or nine T-residues are substituted with 6′-OH-dT residues (Table 12). These two compounds are used for inhibition of the replication of the hybrid virus containing a replicase part of the non-cytotoxic SFV mutant with inserted marker and HCV fragment with respective target site (NS3-4B region) as described previously.

TABLE 12

| | | % of 6- | % of 6- | |
|---|---|---|---|---|
| | Number | OH-dU | OH-dU | |
| | of 6-OH- | residues | residues | Sequence of the |
| | dU | (from | (from | inhibitor, 5′ to |
| Name of | residues in | total T | total | 3′ U indicates |
| compound | compound | residues) | residues) | the modified base |
| HMO_11 | 0 | 0 | 0 | AGTTGTCTCCTGCCTGCTTAGTC |
| | | | | (SEQ ID NO. 26) |
| HMO_12 | 9 | 100 | 39 | AGUUGUCUCCUGCCUGCUUAGUC |
| | | | | (SEQ ID NO. 27) |

### Example 9

### Comparison of the Antiviral Effects of Oligonucleotides Containing Multiple Hydroxynucleobases or Mercaptonucleobases at the Same Positions

To determine the effect of mercaptonucleobases on the inhibitory activity of antiviral antisense oligos, oligonucleotides having multiple 5-SH-dC or 8-5H-dG bases per oligonucleotide are generated and antiviral activity is compared to oligonucleotides containing hydroxynucleobases at the corresponding positions. An inhibition assay measuring HCV replication by modified oligonucleotides containing seven 5-SH-dC residues or five 8-5H-dG residues is carried out. The inhibitors are antisense to the region of NS3 of HCV 1b genotype and comprise the sequence: 5′ AGTTGTCTCCTGCCTGCTTAGTC 3′ (SEQ ID NO. 26) and having zero modified residues, seven 5-SH-dC bases or 5-OH-dC residues (substituting all C-residues), or five 8-5H-dG or 8-oxo-dG residues (substituting all G-residues). These five compounds (Table 13) are used for inhibition of the replication of the hybrid virus containing a replicase part of the non-cytotoxic SFV mutant with inserted marker and HCV fragment with respective target site (NS3-4B region) as described above.

TABLE 13

| | | Number of | | Sequence of the |
|---|---|---|---|---|
| | Number | 8-SH-dG | % of | inhibitor, 5′ to 3′ |
| | of 5-SH- | residues | modified | Y indicates 5-SH-dC, |
| | dC or 5- | or 8-oxo- | residues | Z indicates 5-OH- |
| | OH-dC | dG | (from | dC, X indicates |
| Name of | residues in | residues in | total | 8-SH-dG and W |
| compound | compound | compound | residues) | indicates 8-oxo-dG |
| HMO_11 | 0 | 0 | 0 | AGTTGTCTCCTGCCTGCTTAGTC |
| | | | | (SEQ ID NO. 26) |
| HMO_13 | 7 | 0 | 30.4 | AGTTGTYTYYTGYYTGYTTAGTY |
| | | | | (SEQ ID NO. 28) |
| HMO_14 | 7 | 0 | 30.4 | AGTTGTZTZZTGZZTGZTTAGTZ |
| | | | | (SEQ ID NO. 29) |
| HMO_15 | 0 | 5 | 21.7 | AXTTXTCTCCTXCCTXCTTAXTC |
| | | | | (SEQ ID NO. 30) |
| HMO_16 | 0 | 5 | 21.7 | AWTTWTCTCCTWCCTWCTTAWTC |
| | | | | (SEQ ID NO. 31) |

Strong antiviral effect of the multiply-substituted oligonucleotides are expected, possibly having improved antiviral activity when mercaptonucleobases are present than when hydroxynucleobases are used.

### Example 10

### Use of Combinations of Multiple Mercaptonucleobases and Hydroxynucleobases Increases Antiviral Efficiency

To determine the effect of multiple mercaptonucleobases and hydroxynucleobases in oligonucleotide anti-viral activity, the use of a combination of mercapto- and hydroxyl-modified nucleobases in one oligonucleotide is assayed. This is illustrated by the example of inhibition of HCV replication by modified oligonucleotides containing five 5-SH-dC and two 5-OH-dC residues or two 5-SH-dC and five 5-OH-dC residues Inhibitors antisense to the region of NS3 of HCV 1b genotype and having the sequence: 5′ AGTTGTCTCCTGCCTGCTTAGTC 3′ (SEQ ID NO. 26) with either zero modified residues, with five 5-SH-dC residues and two 5-OH-dC residues, or having two 5-SH-dC and five 5-OH-dC residues (thus substituting all C-residues) were used. These three compounds together with HMO_{—}13 and HMO_{—}14 (Table 14) are used for inhibition of the replication of the hybrid virus containing a replicase part of the non-cytotoxic SFV mutant with inserted marker and HCV fragment with respective target site (NS3-4B region) as above.

TABLE 14

| | | | % of | |
|---|---|---|---|---|
| | Numbero f | Number of | modified | |
| | 5-SH-dC | 5-0H-dC | residues | Sequence of the |
| | residues | residues | (from | inhibitor, 5′ to 3′ |
| Name of | in | in | total | Y indicates 5-SH-dC, |
| compound | compound | compound | residues) | Z indicates 5-OH-dC |
| HMO_11 | 0 | 0 | 0 | AGTTGTCTCCTGCCTGCTTAGTC |
| | | | | (SEQ ID NO. 26) |
| HMO_13 | 7 | 0 | 30.4 | AGTTGTYTYYTGYYTGYTTAGTY |
| | | | | (SEQ ID NO. 32) |
| HMO_14 | 7 | 0 | 30.4 | AGTTGTZTZZTGZZTGZTTAGTZ |
| | | | | (SEQ ID NO. 33) |
| HMO_17 | 5 | 2 | 30.4 | AGTTGTYTYZTGYZTGYTTAGTY |
| | | | | (SEQ ID NO. 34) |
| HMO_18 | 2 | 5 | 30.4 | AGTTGTZTZYTGZYTGZTTAGTZ |
| | | | | (SEQ ID NO. 35) |

Additional assays are carried out using modified oligonucleotide inhibitors antisense to the region of NS3 of HCV 1b genotype and having the sequence: 5′ AGTTGTCTCCTGCCTGCTTAGTC 3, containing seven 5-SH-dC and five 8-5H-dG residues, seven 5-OH-dC and five 5-oxo-dG residues or nine 6-OH-dU and seven 5-OH-dC residues (Table 15). Thus oligonucleotides are used which substitute all C-residues and G-residues with modified nucleotides or substituting all T- and C-nucleotides. These four compounds together with HMO_{—}13, HMO_{—}14, HMO_{—}15 and HMO_{—}16 (previously described) are used for inhibition of the replication of the hybrid virus as described above.

TABLE 15

| | | Number | | Sequence of the |
|---|---|---|---|---|
| | | of 8-SH- | | inhibitor, 5′ to 3′ |
| | Number | dG | Number | Y indicates 5-SH-dC, |
| | of 5-SH- | residues | of | Z indicates 5-OH-dC, |
| | dC or 5- | or 8-oxo- | modified | X indicates 8-SH-dG, |
| | OH-dC | dG | 6-OH-dU | W indicates 8-oxo- |
| Name of | residues in | residues in | residues in | dG and U indicated |
| compound | compound | compound | compound | 6-OH-dU residues |
| HMO_11 | 0 | 0 | 0 | AGTTGTCTCCTGCCTGCTTAGTC |
| | | | | (SEQ ID NO. 26) |
| HMO_13 | 7 | 0 | 0 | AGTTGTYTYYTGYYTGYTTAGTY |
| | | | | (SEQ ID NO. 28) |
| HMO_14 | 7 | 0 | 0 | AGTTGTZTZZTGZZTGZTTAGTZ |
| | | | | (SEQ ID NO. 29) |
| HMO_15 | 0 | 5 | 0 | AXTTXTCTCCTXCCTXCTTAXTC |
| | | | | (SEQ ID NO. 30) |
| HMO_16 | 0 | 5 | 0 | AWTTWTCTCCTWCCTWCTTAWTC |
| | | | | (SEQ ID NO. 31) |
| HMO_19 | 7 | 5 | 0 | AWTTWTZTZZTWZZTWZTTAWTZ |
| | | | | (SEQ ID NO. 35) |
| HMO_20 | 7 | 5 | 0 | AXTTXTYTYYTXYYTXYTTAXTY |
| | | | | (SEQ ID NO. 36) |
| HMO_21 | 7 | 0 | 9 | AGUUGUZUZZUGZZUGZUUAGUZ |
| | | | | (SEQ ID NO. 37) |

A strong antiviral effect is expected, possibly better in the oligos expressing mercaptonucleobases than those having hydroxynucleobases. The previous results suggest that any type of modifications (mercapto/hydroxyl and/or different nucleobases) can be combined and this will result in enhanced antiviral effects. Any number of possible combinations are contemplated.

### Example 11

### Nuclease Complex Increases Antiviral Efficiency of Oligonucleotides with any Modified Nucleobase and any Combination of Those

Addition of a nuclease complex to modified oligonucleotides has been shown to increase the gene inactivation of modified oligonucleotides (see WO 2007/125173). To determine the effects of nuclease complexed to modified oligonucleotides, inhibitors antisense to the region of NS3 of HCV 1b genotype and having sequence: 5′ AGTTGTCTCCTGCCTGCTTAGTC 3′ (SEQ ID NO. 26) are assayed. Oligonucleotides HMO_{—}11 (with no modifications), HMO_{—}12, HMO_{—}13, HMO_{—}14, HMO_{—}15, HMO_{—}16, HMO_{—}17, HMO_{—}18, HMO_{—}19, HMO_{—}20 and HMO_{—}21 (Table 16), with or without nuclease complex, are used for inhibition of the replication of the hybrid virus.

TABLE 16

| | | Number | | |
|---|---|---|---|---|
| | | of 8-SH- | | Sequence of the |
| | Number | dG | Number | inhibitor, 5′ to 3′ |
| | of 5-SH- | residues | of | Y indicates 5-SH-dC, |
| | dC or 5- | or 8-oxo- | modified | Z indicates 5-OH-dC, |
| | OH-dC | dG | 6-OH-dU | X indicates 8-SH-dG, |
| | residues | residues | residues | W indicates 8-oxo- |
| Name of | in | in | in | dG and U indicated |
| compound | compound | compound | compound | 6-OH-dU residues |
| HMO_11 | 0 | 0 | 0 | AGTTGTCTCCTGCCTGCTTAGTC |
| | | | | (SEQ ID NO. 26) |
| HMO_12 | 0 | 0 | 9 | AGUUGUCUCCUGCCUGCUUAGUC |
| | | | | (SEQ ID NO. 38) |
| HMO_13 | 7 | 0 | 0 | AGTTGTYTYYTGYYTGYTTAGTY |
| | | | | (SEQ ID NO. 39) |
| HMO_14 | 7 | 0 | 0 | AGTTGTZTZZTGZZTGZTTAGTZ |
| | | | | (SEQ ID NO. 40) |
| HMO_15 | 0 | 5 | 0 | AXTTXTCTCCTXCCTXCTTAXTC |
| | | | | (SEQ ID NO. 41) |
| HMO_16 | 0 | 5 | 0 | AWTTWTCTCCTWCCTWCTTAWTC |
| | | | | (SEQ ID NO. 42) |
| HMO_17 | 7 | 0 | 0 | AGTTGTYTYZTGYZTGYTTAGTY |
| | | | | (SEQ ID NO. 43) |
| HMO_18 | 7 | 0 | 0 | AGTTGTZTZYTGZYTGZTTAGTZ |
| | | | | (SEQ ID NO. 44) |
| HMO_19 | 7 | 5 | 0 | AWTTWTZTZZTWZZTWZTTAWTZ |
| | | | | (SEQ ID NO. 45) |
| HMO_20 | 7 | 5 | 0 | AXTTXTYTYYTXYYTXYTTAXTY |
| | | | | (SEQ ID NO. 46) |
| HMO_21 | 7 | 0 | 9 | AGUUGUZUZZUGZZUGZUUAGUZ |
| | | | | (SEQ ID NO. 47) |

It is expected that any highly active compound can be additionally activated by the presence of nuclease due to the additive effect of nuclease complex on highly active antisense oligonucleotides with any combination and number of modified nucleobases.

### Example 12

### Use of Combinations of Different Modified Oligonucleotides Increases the Anti-Viral Effect of Such Treatment

The huh7 cells are treated with differently modified oligonucleotides targeted to the same site (samples are selected from compounds HMO_{—}11 to HMO_{—}21 as described previously) or to different target sites: one compound is selected from the list of compounds HMO_{—}11 to HMO-21 and another from list HMO_{—}8 to HMO_{—}10. Combinations of oligonucleotides wherein none of the compounds, either of the compounds or both of the compounds contain a nuclease group are tested. The amount of oligonucleotides used for treatment are as follows: 15 picomols of each (together 30 picomoles) or amounts corresponding to effective dose 50 for each of the compounds.

Combinations of compounds are used for inhibition of the replication of the hybrid virus, as in the assays described above. It is expected that the use of oligonucleotides targeted to different sites is more effective than the use of differently modified oligonucleotides targeted to the same site, and that inhibitory effects of treatment with modified oligonucleotides is increased by use of several different oligonucleotides. Also, treatment with the modified oligonucleotides (by analogy with siRNA treatments described in the art) will reduce the appearance of viral genomes carrying mutations in target regions and thus decrease the resistance to the antiviral treatments.

### Example 13

### Modified Oligonucleotides Inhibit Replication of HCV in Stably Transfected Cell Lines

To determine the effects of modified olgionucletotides in an in vitro model of HCV infection, HCV-persistent infection is modeled by transfection of huh7 cell lines with HCV replicons carrying an antibiotic resistance marker and luciferase of renilla luciferase reporter genes. HCV replication is monitored by analysis of reporter protein expression levels. Cells are treated with modified antisense oligonucleotides (if needed, multiple times) and their efficiency monitored by measurement of luciferase activity at selected timepoints post-treatment.

The assay is similar to those set out above using the oligonucleotide compositions described in the previous examples. The assay is carried out using oligonucleotides having mercaptonucleobases, hydroxynucleobases, the compositions complexed with nucleases using chelating agents and metal ions. It is expected that the compounds will also have an effect against viruses which have established chronic infection (thus it is relevant to the chronic infection of HCV in patients which is the most important disease condition caused by HCV.

### Example 14

### Modified Antisense Oligonucleotides Suppress Expression of HCV Transgenes in Transiently Transfected Mice

HCV does not replicate in rodents and there is no good small animal model for the virus. To demonstrate the efficiency of modified antisense oligonucleotides in in vivo conditions, mice transfected with hydrodynamic shock method were used as a model. The expression constructs, transcribing mRNAs containing coding region of destabilized renilla luciferase reporter and HCV region(s) containing target site(s) for antisense modified oligonucleotides are constructed. The DNA plasmid containing that expression unit are delivered into the liver of subject mice by using the hydrodynamic shock method (Yeikilis et al., World J. Gastroenterol. 12:6149-55. (2006); Andrianaivo et al., J Gene Med. 6:877-83 (2004)) and expression of the HCV sequence-containing mRNAs is quantified by measurement of the reporter activity in liver tissue

Modified antiviral oligonucleotides antisense to the HCV-specific region in the expression construct are delivered to the liver of the mouse using either co-transfection with the expression construct or repeated delivery of the compound before, together and/or after the delivery of expression construct. The compounds described in Examples 3-11 as well as their combinations described in Example 12 are used in the assay.

It is expected that the modified oligonucleotides exhibit effective antiviral activity and inhibit replication of the viral transgene in vivo.

Modified oligonucleotides are also measured against other therapeutically relevant viruses using appropriate animal models well-known in the art.

Numerous modifications and variations in the invention as set forth in the above illustrative examples are expected to occur to those skilled in the art. Consequently only such limitations as appear in the appended claims should be placed on the invention.

1. A method of inhibiting replication of a virus in a subject comprising administering to the subject a composition comprising an oligonucleotide having from 5 to 150 nucleobases, wherein at least one of the nucleobases is a mercapto-modified tautomeric or ionic base (mercaptonucleobase) or a hydroxyl-modified tautomeric or ionic base (hydroxynucelobase).

2. (canceled)

3. A method of inhibiting translation of a target nucleic acid comprising contacting the target nucleic acid with a composition comprising an oligonucleotide under conditions that permit hybridizing of the oligonucleotide to the target nucleic acid, wherein the hybridized oligonucleotide inhibits translation of the target nucleic acid, wherein the target nucleic acid is associated with viral replication, and wherein the oligonucleotide comprises at least one modified nucleobase selected from the group consisting of a mercapto-modified tautomeric or ionic base (mercaptonucleobase) and a hydroxy-modified tautomeric or ionic base (hydroxynucleobase).

4. (canceled)

5. A method of inhibiting replication of a viral genome in a subject, comprising
predicting or determining a nucleotide sequence of a target nucleic acid in a subject or a viral pathogen, wherein the target nucleic acid is associated with viral replication, and
administering to the subject a composition comprising an oligonucleotide having from 5 to 150 nucleobases, wherein at least one of the nucleobases is a mercapto-modified tautomeric or ionic base (mercaptonucleobase) or a hydroxy-modified tautomeric or ionic base (hydroxynucleobase), and wherein under physiological conditions of the subject, said compound is sufficiently complementary to the nucleotide sequence of the target sequence to hybridize thereto in the subject and inhibit viral replication.

6. (canceled)

7. A compound that comprises an oligonucleotide having from 5 to 150 nucleobases,
wherein the oligonucleotide comprises a nucleotide sequence that is at least 80% complementary to a nucleotide sequence from a virus,
wherein at least one of the nucleobases is a mercapto-modified tautomeric or ionic base (mercaptonucleobase) or a hydroxyl-modified tautomeric or ionic base (hydroxynucelobase).

8. The method of claim 1, wherein the oligonucleotide comprises at least one mercaptonucleobase.

9. The method of claim 8, wherein the at least one mercaptonucelobase is selected from the group consisting of 5-mercaptocytosine, 5-mercaptouracil, 8-mercaptoguanine and 8-mercaptoadenine.

10. The method of claim 1, wherein the oligonucleotide comprises at least one hydroxynucleobase.

11. The method of claim 10, wherein the at least one hydroxynucleobase is selected from the group consisting of 5-hydroxycytosine, 5-hydroxyuracil, 8-hydroxyadenine and 8-hydroxyguanine.

12. The method of claim 1, wherein the oligonucleotide further comprises an organic nuclease attached thereto.

13. The method of claim 12, wherein the organic nuclease comprises a chelating organic moiety complexed with a lanthanide metal.

14. The method of claim 13, wherein the lanthanide metal is selected from the group consisting of lanthanum, cerium, praseodymium, neodymium, promethium, samarium, europium, gadolinium, terbium, dysprosium, holmium erbium, thulium, ytterbium, and lutetium.

15. The method of claim 14, wherein the metal is Europium.

16. The method of claim 1, wherein the virus is a fast acute virus.

17. The method of claim 1, wherein the virus is a chronic virus.

18. The method of claim 1, wherein the virus is a positive-strand RNA virus.

19. The method of claim 18, wherein the virus is an alphavirus.

20. The method of claim 18, wherein the virus is Semliki Forest virus (SFV).

21. The method of claim 18, wherein the virus is hepatitis C virus.

22. The method of claim 1, wherein the virus is a DNA virus.

23. The method of claim 22, wherein the virus is a papilloma virus.

24. The method of claim 23, wherein the papilloma virus is selected from the group consisting of bovine papilloma virus 1 and human papilloma virus.

25. The method of claim 22, wherein the oligonucleotide hybridizes to a viral gene encoding a transcription or regulatory factor, and inhibits replication of a virus with a DNA genome.

26. The method of claim 22, wherein the oligonucleotide hybridizes to a viral replication factor and inhibits replication of a virus with a DNA genome.

27. The method of claim 1, wherein the oligonucleotide hybridizes to a virus using reverse transcription in its replication cycle.

28. The method of claim 27, wherein the virus is a retrovirus.

29. The method of claim 28, wherein the retrovirus is human immunodeficiency virus 1.

30. The method of claim 27, wherein the oligonucleotide hybridizes to a viral gene encoding a transcription or a regulatory factor to inhibit replication of a virus replicating by use of reverse transcription.

31. The method of claim 1, wherein the oligonucleotide is from 10-100 nucleobases in length.

32. The method of claim 1, wherein the oligonucleotide is from 10-50 nucleobases in length.

33. The method of claim 1, wherein the oligonucleotide is from 10-30 nucleobases in length.

34. The method of claim 1, wherein the oligonucleotide is from 20-30 nucleobases in length.

35. The method of claim 34, wherein the oligonucleotide is from 21 to 23 nucleobases in length.

36. The method of claim 1, wherein 1% to 100% of nucleobases in the oligonucleotide are modified nucleobases.

37. The method of claim 36, wherein 10& to 90% of the nucleobases are mercapto-modified or hydroxy-modified nucleobases.

38. The method of claim 36, wherein 20% to 80% of the nucleobases are mercapto-modified or hydroxy-modified nucleobases.

39. (canceled)

40. The method or use or compound of claim 36, wherein 40% to 60% of the nucleobases are mercapto-modified or hydroxy-modified nucleobases.

41. The method of claim 36, wherein 50% of the nucleobases are mercapto-modified or hydroxy-modified nucleobases.

42. The method of claim 1, wherein the oligonucleotide contains at least one mercapto-modified nucleobase and at least one hydroxyl-modified nucleobase.

43. The method of claim 1, wherein the oligonucleotide hybridizes to a host factor associated with viral replication, and inhibits viral replication.

44. The method of claim 1, wherein the oligonucleotide hybridizes to a non-coding region of a viral genome.

45. The method of claim 1, wherein the oligonucleotide hybridizes to a coding region of a viral genome.

46. The method of claim 45, wherein the oligonucleotide hybridizes to the coding region of an RNA virus.

47. The method of claim 1, wherein at least two of the oligonucleotides, having different sequences, are administered to the subject, wherein the at least two oligonucleotides hybridize to different target sequences.

48. (canceled)

49. A composition that comprises two compounds of claim 7 in admixture, wherein the oligonucleotides hybridize to different target sequences.

50. A composition according to claim 49, further comprising a pharmaceutically acceptable carrier.

51. The method according to claim 47, wherein the at least two oligonucleotides are specific for different target sequences in the same viral genome.

52. The method of claim 47, wherein the at least two oligonucleotides are specific for target sequences in the same functional unit.

53. The method of claim 47, wherein the at least two oligonucleotides are specific for target sequences in different functional units.

54. The method of claim 1, wherein the composition further comprises a pharmaceutical carrier or excipient.

55. A composition that comprises a compound of claim 7 and a pharmaceutically acceptable carrier.

56. (canceled)

57. (canceled)

58. (canceled)

59. (canceled)

60. (canceled)

61. (canceled)

62. The method of claim 1, wherein the subject is a mammal.

63. The method of claim 62, wherein the subject is human.

64. The method of claim 1, wherein the oligonucleotide is in a liposome.

65. The method of claim 1, wherein hybridizing of the oligonucleotide to a target sequence induces cleavage of the target nucleic acid.

66. (canceled)

67. The method of claim 1, wherein the sequence of the oligonucleotide exhibits at least 90% sequence identity to the target nucleic acid or its complement.

68. The compound of claim 7, wherein the oligonucleotide comprises a nucleotide sequence that is at least 90% complementary to a nucleotide sequence from a virus.

69. A method of determining the effect of the modified oligonucleotide of claim 1 on viral gene replication in vitro comprising:
transfecting a cell with the modified oligonucleotide, and
determining a reduction in viral gene replication relative to a cell infected with the virus and transfected with an oligonucleotide that does not comprise the mercaptonucleohase or the hydroxynucleobase.

70. The method of claim 69, wherein the cell is infected with a virus at a selected multiplicity of infection (moi) of about 0.05.
